(19) United States
(12) Patent Application Publication
(10) Pub. No.: US 2009/0271895 A1
(43) Pub. Date: Oct. 29, 2009

(54) Plants having improved growth characteristics and method for making the same

(75) Inventors: Valerie Frankard, Waterloo (BE); Vladimir Mironov, Gent (BE)

Correspondence Address: CONNOLLY BOVE LODGE & HUTZ, LLP, P O BOX 2207, WILMINGTON, DE, 19899, US

(73) Assignee: CROPDESIGN N.V., ZWIJNAARDE (BE)

(21) Appl. No.: 11/632,580

(22) PCT Filed: Jul. 14, 2005
(86) PCT No.: PCT/EP05/53398

§ 371 Date: Nov. 12, 2007

Related U.S. Application Data
(60) Provisional application No. 60/589,765, filed on Jul. 21, 2004.

(30) Foreign Application Priority Data
Jul. 16, 2004 (EP) 04103421.6

(51) Int. Cl. C12N 15/82; A01H 5/00; C12N 5/04; C07H 21/04; C12Q 1/68

(52) U.S. Cl. 800/290; 800/278; 800/298; 435/419; 536/23.6; 435/6

(57) ABSTRACT

The present invention concerns a method for improving growth characteristics of plants by increasing activity and/or expression in a plant of an SnRK2 kinase or a homologue thereof. One such method comprises introducing into a plant an SnRK2 nucleic acid molecule or functional variant thereof. The invention also relates to transgenic plants having improved growth characteristics, which plants have modulated expression of a nucleic acid encoding an SnRK2 kinase. The present invention also concerns constructs useful in the methods of the invention.

The present invention relates generally to the field of molecular biology and concerns a method for improving plant growth characteristics. More specifically, the present invention concerns a method for increasing yield and/or biomass of a plant by increasing the activity of an SNF1 related protein kinase (SnRK2) or a homologue thereof in a plant. The present invention also concerns plants having increased expression of a nucleic acid encoding an SnRK2 protein kinase or a homologue thereof, which plants have improved growth characteristics relative to corresponding wild type plants. The invention also provides constructs useful in the methods of the invention.

Given the ever-increasing world population, and the dwindling area of land available for agriculture, it remains a major goal of agricultural research to improve the efficiency of agriculture and to increase the diversity of plants in horticulture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic complements that may not always result in the desirable trait being passed on from parent plants. Advances in molecular biology have allowed mankind to manipulate the germplasm of animals and plants. Genetic engineering of plants entails the isolation and manipulation of genetic material (typically in the form of DNA or RNA) and the subsequent introduction of that genetic material into a plant Such technology has led to the development of plants having various improved economic, agronomic or horticultural traits. Traits of particular economic interest are growth characteristics such as high yield. Yield is normally defined as the measurable produce of economic value from a crop. This may be defined in terms of quantity and/or quality. Yield is directly dependent on several factors, for example, the number and size of the organs, plant architecture (for example, the number of branches), seed production and more. Root development, nutrient uptake and stress tolerance may also be important factors in determining yield. Crop yield may therefore be increased by optimising one of the abovementioned factors.

The yeast protein kinase SNF1 is reportedly involved in the response to glucose starvation stress. It supposedly takes part in activating genes that are repressed by glucose by phosphorylating the repressor protein Mig1. SNF1 has orthologues in other organisms such as the AMP-activated protein kinase (AMPK) in mammals. AMPK becomes activated by increased 5′-AMP concentrations as a result of ATP depletion, which may be caused by stress conditions, including heat shock or glucose starvation. Plants also have SNF1-related kinases, named SnRKs. Plant SnRKs are divided in three subgroups, SnRK1 to SnRK3. The SnRK1 subgroup is most closely related to SNF1, both structurally and functionally; whereas the subgroups SnRK2 and SnRK3 may be unique to plants. The SnRK2 proteins lack the C-terminal regulatory domain found in SNF1, but instead have at their C-terminus an acidic stretch of glutamic and aspartic acids. SnRK2 proteins have a molecular weight of around 40 kDa and are encoded by a small gene family: both Arabidopsis and rice have been reported to have 10 SnRK2 genes. The first plant SNF1-related protein kinase 2 (SnRK2), designated PKABA1, was isolated by Anderberg and Walker-Simmons (Proc. Natl. Aced. Sci. USA 89, 10183-10187, 1992). It was found to be induced by abscisic acid (ABA) and dehydration. Later, related proteins were isolated, such as ASK1 and ASK2, (Park et al., Plant Molecular Biology 22, 615-624, 1993). These genes were reported to be expressed in several plant organs, but were most abundant in leaves. Another member of the SnRK2 subgroup is OST1 (Mustilli et al., Plant Cell 14, 3089-3099, 2002). OST1 was expressed in stomatal guard cells and vascular tissue, and was postulated to act between perception of abscisic acid (ABA) and production of reactive oxygen species that elicits stomatal closure. In rice, all the SnRK2 proteins were found to be activated by hyperosmotic stress and some of them were also activated by ABA (Kobyashi et al., Plant Cell 16, 1163-1177, 2004). REK (renamed SAPK3, Kobyashi at al., 2004) was reported to be expressed in leaves and maturing seeds, but not in stems or roots. Recombinant REK proteins showed increased autophosphorylation activity in the presence of Ca²⁺.

WO 98/05760 discloses more than 20 nucleotide sequences encoding proteins involved in phosphorus uptake and metabolism (psr proteins). One of these psr proteins is the protein kinase psrPK, a protein related to SnRK2 which is expressed upon phosphate starvation. It was speculated that this protein and other psr proteins would be useful in manipulating phosphorus metabolism, however none of the proposed phenotypes, many of them relating to increased stress resistance, were enabled. Assmann and Li (WO 01/02541) described the protein kinase AAPK, another relative of SnRK2. Loss of function of AAPK was reported to reduce sensitivity to abscisic acid-induced stomatal closure. It was therefore suggested that the opposite, (increased expression or increased activity of AAPK) would result in plants with increased drought stress resistance. The authors however did not show that this was indeed the case. So far the available experimental data for SnRK2-related proteins mainly suggested a role in stress responses of plants.

None of the prior art documents has demonstrated or suggested that increased expression or increased activity and/or expression of an SnRK2 protein results in yield increase, relative to corresponding wild type and unstressed plants.

It has now surprisingly been found that increasing activity and/or expression of an SnRK2 protein in plants results in plants having improved growth characteristics, and in particular yield, relative to corresponding wild type plants. These results were obtained under standard plant growth conditions, and the yield increase is not the consequence of increased stress resistance.

Structurally, SnRK2 proteins are serine/threonine protein kinases, with a catalytic domain that is classified in the SMART database as an S_TKc type (SMART Accession number SM00220). The active site corresponds to the PROSITE signature, PS00108 (Prosite, Swiss Institute of Bioinformatics, http://us.expasy.org): [LIVMFYC]-x-[HY]-x-D-[LIVMFY]-K-x(2)-N-[LIVMFYCT](3)

The C-terminal part comprises a stretch of poly (Glu and/or Asp) residues of unknown function.

According to one embodiment of the present invention there is provided a method for improving growth characteristics of a plant comprising increasing activity and/or expression in a plant of an SnRK2 polypeptide or a homologue thereof and optionally selecting for plants having improved growth characteristics.

Advantageously, performance of the method according to the present invention results in plants having a variety of improved growth characteristics, such as improved growth, improved yield, improved biomass, improved architecture or improved call division, each relative to corresponding wild type plants. Preferably, the improved growth characteristics comprise at least increased yield relative to corresponding wild type plants. Preferably, the increased yield is increased biomass and/or increased seed yield, which includes one or more of increased number of (filled) seeds, increased total weight of seeds, increased thousand kernel weight and increased harvest index. It should be noted that the yield increase is not the consequence of increased stress resistance.

The term “increased yield” as defined herein is taken to mean an increase in any one or more of the following, each relative to corresponding wild type plants: (i) increased biomass (weight) of one or more parts of a plant, particularly aboveground (harvestable) parts, increased root biomass or increased biomass of any other harvestable part; (ii) increased total seed yield, which includes an increase in seed biomass (seed weight) and which may be an increase in the seed weight per plant or on an individual seed basis; (iii) increased number of (filled) seeds; (iv) increased seed size; (v) increased seed volume; (vi) increased individual seed area; (vii) increased individual seed length; (viii) increased harvest index, which is expressed as a ratio of the yield of harvestable parts, such as seeds, over the total biomass; (ix) increased number of florets per panicle which is extrapolated from the total number of seeds counted and the number of primary panicles; and (x) increased thousand kernel weight (TKW). which is extrapolated from the number of filled seeds counted and their total weight. An increased TKW may result from an increased seed size (length, width or both) and/or seed weight. An increased TKW may result from an increase in embryo size and/or endosperm size.

Taking corn as an example, a yield increase may be manifested as one or more of the following: increase in the number of plants per hectare or acre, an increase in the number of ears per plant, an increase in the number of rows, number of kernels per row, kernel weight, TKW, ear length/diameter, among others. Taking rice as an example, a yield increase may be manifested by an increase in one or more of the following: number of plants per hectare or acre, number of panicles per plant, number of spikelets per panicle, number of flowers per panicle, increase in the seed filling rate, increase in TKW, among others. An increase in yield may also result in modified architecture, or may occur as a result of modified architecture.

Preferably, performance of the methods according to the present invention results in plants having increased yield and more particularly, increased biomass and/or increased seed yield. Preferably, the increased seed yield comprises an increase in one or more of number of (filled) seeds, total seed weight, seed size, thousand kernel weight and harvest index, each relative to control plants. Therefore, according to the present invention, there is provided a method for increasing plant yield, which method comprises increasing activity and/or expression in a plant of an SnRK2 polypeptide or a homologue thereof.

Since the improved plants according to the present invention have increased yield, it is likely that these plants exhibit an increased growth rate (during at least part of their life cycle), relative to the growth rate of corresponding wild type plants at a corresponding stage in their life cycle. The increased growth rate may be specific to one or more parts or call types of a plant (including seeds), or may be throughout substantially the whole plant. Plants having an increased growth rate may have a shorter life cycle. The life cycle of a plant may be taken to mean the time needed to grow from a dry mature seed up to the stage where the plant has produced dry mature seeds, similar to the starting material. This life cycle may be influenced by factors such as early vigour, growth rate, flowering time and speed of seed maturation. An increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible. If the growth rate is sufficiently increased, it may allow for the sowing of further seeds of the same plant species (for example sowing and harvesting of rice plants followed by sowing and harvesting of further rice plants all within one conventional growing period). Similarly, if the growth rate is sufficiently increased, it may allow for the sowing of further seeds of different plants species (for example the sowing and harvesting of rice plants followed by, for example, the sowing and optional harvesting of soy bean, potatoes or any other suitable plant). Harvesting additional times from the same rootstock in the case of some plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves plotting growth experiments, such parameters may be: T-Mid (the time taken for plants to reach 50% of their maximal size) and T-90 (time taken for plants to reach 90% of their maximal size), amongst others.

Performance of the methods of the invention gives plants having an increased growth rate. Therefore, according to the present invention, there is provided a method for increasing the growth rate of plants, which method comprises increasing activity and/or expression in a plant of an SnRK2 polypeptide or a homologue thereof.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the typical stresses to which a plant may be exposed. These stresses may be the everyday biotic and/or abiotic (environmental) stresses to which a plant is exposed. Typical abiotic or environmental stresses include temperature stresses caused by atypical hot or cold/freezing temperatures; salt stress; water stress (drought or excess water). Abiotic stresses may also be caused by chemicals. Biotic stresses are typically those stresses caused by pathogens, such as bacteria, viruses, fungi and insects.

The abovementioned growth characteristics may advantageously be improved in any plant.

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest or the genetic modification in the gene/nucleic add of interest. The term “plant” also encompasses plant cells, suspension cultures, callus tissue, embryos, meristematic regions, gametophytes, sporophytes, pollen, and microspores, again wherein each of the aforementioned comprise the gene/nucleic acid of interest.

Plants that are particularly useful in the methods of the invention include algae, ferns, and all plants which belong to the superfamily Viridiplantae, in particular monocotyledonous and dicotyledonous plants, including fodder or forage legumes, ornamental plants, food crops, trees, or shrubs selected from the list comprising Abelmoschus spp., Acer spp., Actinidia spp., Agropyron spp., Allium spp., Amaranthus spp., Ananas comosus, Annona spp., Apium graveolens, Arabidopsis thaliana, Arachis spp, Artocarpus spp., Asparagus officinalis, Avena sativa, Averrhoa carambola, Benincasa hispida, Bertholletia excelsea, Beta vulgaris, Brassica spp., Cadaba farinosa, Camellia sinensis, Canna indica, Capsicum spp., Carica papaya, Carissa macrocarpa, Carthamus tinctorius, Carya spp., Castanea spp., Cichorium endivia, Cinnamomum spp., Citrullus lanatus, Citrus spp., Cocos spp., Coffea spp., Cola spp., Colocasia esculenta, Corylus spp., Crataegus spp., Cucumis spp., Cucurbita spp., Cynara spp., Daucus carota, Desmodium spp., Dimocarpus longan, Dioscorea spp., Diospyros spp., Echinochloa spp., Eleusine coracana, Eriobotrya japonica, Eugenia uniflora, Fagopyrum spp., Fagus spp., Ficus carica, Fortunella spp., Fragaria spp., Ginkgo biloba, Glycine spp., Gossypium hirsutum, Helianthus spp., Hibiscus spp., Hordeum spp., Ipomoea batatas, Juglans spp., Lactuca sativa, Lathyrus spp., Lemna spp., Lens culinaris, Linum usitatissimum, Litchi chinensis, Lotus spp., Luffa acutangula, Lupinus spp., Macrotyloma spp., Malpighia emarginata, Malus spp., Mammea americana, Mangifera indica, Manihot spp., Manilkara zapota, Medicago sativa, Melilotus spp., Mentha spp., Momordica spp., Morus nigra, Musa spp., Nicotiana spp., Olea spp., Opuntia spp., Ornithopus spp., Oryza spp., Panicum miliaceum, Passiflora edulis, Pastinaca sativa, Persea spp., Petroselinum crispum, Phaseolus spp., Phoenix spp., Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Rubus spp., Saccharum spp., Sambucus spp., Secale cereale, Sesamum spp., Solanum spp., Sorghum bicolor, Spinacia spp., Syzygium spp., Tamarindus indica, Theobroma cacao, Trifolium spp., Triticosecale rimpaui, Triticum spp., Vaccinium spp., Vicia spp., Vigna spp., Vitis spp., Zea mays, Zizania palustris, Ziziphus spp., amongst others.

According to a preferred feature of the present invention, the plant is a crop plant comprising soybean, sunflower, canola, alfalfa, rapeseed or cotton. Further preferably, the plant according to the present invention is a monocotyledonous plant such as sugarcane, most preferably a cereal, such as rice, maize, wheat, millet, barley, rye, oats or sorghum.

The activity of an SnRK2 protein may be increased by increasing levels of the SnRK2 polypeptide. Alternatively, activity may also be increased when there is no change in levels of an SnRK2, or even when there is a reduction in levels of an SnRK2. This may occur when the intrinsic properties of the polypeptde are altered, for example, by making a mutant or selecting a variant that is more active that the wild type.

The term “SnRK2 or homologue thereof” as defined herein refers to a polypeptide comprising (i) a functional serine/threonine kinase domain, (ii) the conserved signature sequence W(F/Y)(L/M/R/T)(K/R)(N/G/R)(L/P/I)(P/L)(A/G/V/R/K/I)(D/E/V) (SEQ ID NO: 6) and (iii) an acidic C-terminal domain that starts from the last residue of SEQ ID NO: 6. The “SnRK2 or homologue thereof” has in increasing order of preference at least 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO: 2. The overall sequence identity is determined using a global alignment algorithm, such as the Needleman Wunsch algorithm in the program GAP (GCG Wisconsin Package, Accalrys).

Furthermore, such “SnRK2 or homologue thereof”, when expressed under control of a GOS2 promoter in the Oryza sativa cultivar Nipponbare, increases aboveground biomass and/or seed yield compared to corresponding wild type plants. This increase in seed yield may be measured in several ways, for example as an increase of thousand kernel weight.

The various structural domains in an SnRK2 protein may be identified using specialised databases e.g. SMART (Schultz et al. (1998) Proc. Natl. Acad. Sci. USA 95, 5857-5864; Letunic et al. (2002) Nucleic Acids Res 30, 242-244; http://smart.embl-heidelberg.de/), InterPro (Mulder et al., (2003) Nucl. Acids. Res. 31, 315-318; http//www.ebi.ac.uk/interpro/), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-6, AAAIPress, Menlo Park; Hulo et al., Nucl. Acids. Res. 32:D134-D137, (2004), http://www.expasy.org/prosite/) or Pfam (Bateman et al., Nucleic Acids Research 30(1):276-280 (2002), http://www.sanger.ac.uk/Software/Pfam/).

The kinase domain of SnRK2 is of a S_TKc type (SMART accession number SM00221, Interpro accession number IPR002290), and is functional in the sense that it has Ser/Thr kinase activity. The predicted active site (ICHRDLKLENTLL, wherein D is the predicted catalytic residue) corresponds to the PROSITE signature PS00108. The ATP binding site (IGAGNFGVARLMKVKNSKELVAMK) corresponds to the PROSITE signature PS00107.

Preferably, the conserved signature sequence of SEQ ID NO: 6 has the sequence: W(F/Y)(L/M/R)K(N/R)(L/I)P(A/G/V/R/K/I)(D/E), more preferably, the conserved signature sequence of SEQ ID NO: 6 has the sequence: W(F/Y)LKNLP(R/K)E; most preferably, the conserved signature sequence of SEQ ID NO: 6 has the sequence: WFLKNLPRE.

The acidic C-terminal domain as used herein is defined as the C-terminal part of the SnRK2 protein starting from the last residue in the conserved signature sequence defined above (D or E in SEQ ID NO: 6), and which C-terminal part has an isoelectric point (pI) ranging between 2.6 and 4.1, preferably between 3.6 and 3.9, most preferably the pI of the acidic C-terminal domain is 3.7. The pI values are calculated using the EMBOSS package (Rice at al., Trends in Genetics 16, 276-277, 2000).

Methods for the search and identification of SnRK2 homologues would be well within the realm of persons skilled in the art. Such methods comprise comparison of the sequences represented by SEQ ID NO: 1 or 2, in a computer readable format, with sequences that are available in public databases such as MIPS (http://mips.gsf.de/), GenBank (http://www.ncbi.nlm.nih.gov/Genbank/index.html) or EMBL Nucleotide Sequence Database (http://www.ebi.ac.uk/embl/index.html), using algorithms well known in the art for the alignment or comparison of sequences, such as GAP (Needleman and Wunsch, J. Mol. Biol. 48; 443-453 (1970)), BESTFIT (using the local homology algorithm of Smith and Waterman (Advances in Applied Mathematics 2; 482-489 (1981))), BLAST (Altschul, S. F., Gish, W., Miller, W., Myers, E. W. & Lipman, D. J., J. Mol. Biol. 215:403-410 (1990)), FASTA and TFASTA (W. R. Pearson and D. J. Lipman Proc. Natl. Acad. Sci. USA 85:2444-2448 (1988)). The software for performing BLAST analysis is publicly available through the National Centre for Biotechnology Information (NCBI). The homologues mentioned below were identified using BLAST default parameters (BLOSUM62 matrix, gap opening penalty 11 and gap extension penalty 1) and preferably full-length sequences are used for analysis.

Examples of proteins falling under the definition of “SnRK2 polypeptide or a homologue thereof” include Arabidopsis proteins and proteins from other species such as rice, soybean and tobacco.

Two special forms of homology, orthologous and paralogous, are evolutionary concepts used to describe ancestral relationships of genes. The term “paralogous” relates to homologous genes that result from one or more gene duplications within the genome of a species. The term “orthologous” relates to homologous genes in different organisms due to ancestral relationship of these genes.

Paralogues of SnRK2 polypeptides may easily be identified by performing a BLAST analysis against a set of sequences from the same species as the query sequence. Orthologues in, for example, monocot plant species may easily be found by performing a so-called reciprocal blast search. This may be done by a first blast involving blasting the sequence in question (for example, SEQ ID NO: 1 or SEQ ID NO: 2, being from Arabidopsis thaliana) against any sequence database, such as the publicly available NCBI database which may be found at: http://www.ncbi.nlm.nih.gov. If orthologues in rice were sought, the sequence in question would be blasted against, for example, the 28,469 full-length cDNA clones from Oryza sativa Nipponbare available at NCBI. BLASTn or tBLASTX may be used when starting from nucleotides or BLASTP or TBLASTN when starting from the protein, with standard default values. The blast results may be filtered. The full-length sequences of either the filtered results or the non-filtered results are then blasted back (second blast) against the sequences of the organism from which the sequence in question is derived, in casu Arabidopsis thaliana. The results of the first and second blasts are then compared. An orthologue is found when the results of the second blast give as hits with the highest similarity a query SnRK2 nucleic acid or SnRK2 polypeptide. If for a specific query sequence the highest hit is a paralogue of SnRK2 then such query sequence is also considered a homologue of SnRK2, provided that this homologue comprises a functional serine/threonine kinase domain, the conserved signature sequence of SEQ ID NO: 6 and an acidic C-terminal region as defined above. In the case of large families, ClustalW may be used, followed by the construction of a neighbour joining tree, to help visualize the clustering.

The term “homologues” as used herein also encompasses paralogues and orthologues of the proteins useful in the methods according to the invention. Paralogues from Arabidopsis include the proteins as given in the GenBank accessions NP-172563, NP_{—}849834 (SEQ ID NO: 8), NP_{—}201170 (SEQ ID NO: 10), NP_{—}196476 (SEQ ID NO: 12), NP_{—}567945 (SEQ ID NO: 14), NP_{—}179885 (SEQ ID NO: 16), NP_{—}201489 (SEQ ID NO: 18), NP_{—}974170 (SEQ ID NO: 20), NP_{—}190619 (SEQ ID NO: 22), NP_{—}195711 (SEQ ID NO: 24). Orthologues and paralogues from rice (induding GenBank accessions BAD17997 (SEQ ID NO: 26), BAD17998 (SEQ ID NO: 28), BAD17999 (SEQ ID NO: 30), BAD18000 (SEQ ID NO: 32), BAD18001 (SEQ ID NO: 34), BAD18002 (SEQ ID NO: 36), BAD18003 (SEQ ID NO: 38), BAD18004 (SEQ ID NO: 40), BAD18005 (SEQ ID NO: 42), BAD18006 (SEQ ID NO: 44)), from B. napus (AAA33003 (SEQ ID NO: 46) and AAA33004 (SEQ ID NO: 48)), from soybean (AAB68961 (SEQ ID NO: 50) and AAB68962 (SEQ ID NO: 52)) and from tobacco (AAL89456 (SEQ ID NO: 54)) were identified using a reciprocal BLAST procedure. Preferably the orthologues and paralogues useful in the present invention have the same structure and activity as SnRK2 and have the highest similarity to SnRK2 as represented by SEQ ID NO: 2 in a reciprocal BLAST search.

It is to be understood that the term SnRK2 polypeptide or a homologue thereof is not to be limited to the sequence represented by SEQ ID NO: 2 or to the homologues listed above, but that any polypeptide meeting the criteria of comprising a functional serine/threonine kinase domain, and the conserved signature sequence of SEQ ID NO: 6 and a C-terminal acidic domain as defined above, and/or being a paralogue or orthologue of SnRK2 or having at least 55% sequence identity to the sequence of SEQ ID NO: 2, may be suitable for use in the methods of the invention.

To determine the kinase activity of SnRK2, several assays are available and well known in the art (for example Current Protocols in Molecular Biology, Volumes 1 and 2, Ausubel et al. (1994), Current Protocols; or online such as http://www.protocol-online.org). In brief, the kinase assay generally involves (1) bringing the kinase protein into contact with a substrate polypeptide containing the target site to be phosphorylated; (2) allowing phosphorylation of the target site in an appropriate kinase buffer under appropriate conditions; (3) separating phosphorylated products from non-phosphorylated substrate after a suitable reaction period. The presence or absence of kinase activity is determined by the presence or absence of a phosphorylated target. In addition, quantitative measurements may be performed.

Purified SnRK2 protein, or cell extracts containing or enriched in the SnRK2 protein could be used as source for the kinase protein. As a substrate, small peptides are particularly well suited. The peptide must comprise one or more serine, threonine, or tyrosine residues in a phosphorylation site motif. A compilation of phosphorylation sites may be found in Biochimica et Biophysica Acta 1314, 191-225, (1996). In addition, the peptide substrates may advantageously have a net positive charge to facilitate binding to phosphocellulose filters, (allowing to separate the phosphorylated from non-phosphorylated peptides and to detect the phosphorylated peptides). If a phosphorylation site motif is not known, a general tyrosine kinase substrate may be used. For example, “Src-related peptide” (RRLIEDAEYAARG) is a substrate for many receptor and non-receptor tyrosine kinases). To determine the kinetic parameters for phosphorylation of the synthetic peptide, a range of peptide concentrations is required. For initial reactions, a peptide concentration of 0.7-1.5 mM may be used. For each kinase enzyme, it is important to determine the optimal buffer, ionic strength, and pH for activity. A standard 5×Kinase Buffer generally contains 5 mg/ml BSA (Bovine Serum Albumin preventing kinase adsorption to the assay tube), 150 mm Tris-C (pH 7.5), 100 mM MgCl₂. Divalent cations are required for most tyrosine kinases, although some tyrosine kinases (for example, insulin-, IGF-1-, and PDGF receptor kinases) require MnCl₂ instead of MgCl₂ (or in addition to MgCl₂). The optimal concentrations of divalent cations must be determined empirically for each protein kinase.

A commonly used donor of the phophoryl group is radio-labelled [gamma-³²P]ATP (normally at 0.2 mM final concentration). The amount of ³²P incorporated in the peptides may be determined by measuring activity on the nitrocellulose dry pads in a scintillation counter.

Alternatively, the activity of an SnRK2 protein or homologue thereof may be assayed by expressing the SnRK2 protein or homologue thereof under control of a GOS2 promoter in the Oryza sativa cultivar Nipponbare, which results in plants with increased aboveground biomass and/or increased seed yield compared to corresponding wild type plants. This increase in seed yield may be measured in several ways, for example as an increase of thousand kernel weight.

The nucleic acid encoding an SnRK2 polypeptide or a homologue thereof may be any natural or synthetic nucleic acid. An SnRK2 polypeptide or a homologue thereof as defined hereinabove is encoded by an SnRK2 nucleic acid molecule. Therefore the term “SnRK2 nucleic acid molecule” or “SnRK2 gene” as defined herein is any nucleic acid molecule encoding an SnRK2 polypeptide or a homologue thereof as defined hereinabove. Examples of SnRK2 nucleic acid molecules include those represented by SEQ ID NO: 1, and those encoding the above mentioned homologues. SnRK2 nucleic acids and functional variants thereof may be suitable in practising the methods of the invention. Functional variant SnRK2 nucleic acids include portions of an SnRK2 nucleic acid molecule and/or nucleic acids capable of hybridising with an SnRK2 nucleic acid molecule. The term “functional” in the context of a functional variant refers to a variant SnRK2 nucleic acid (i.e. a portion or a hybridising sequence), which encodes a polypeptide comprising a functional kinase domain, the conserved signature sequence of SEQ ID NO: 6 and an acidic C-terminal domain as defined above.

The SnRK2 type kinases in plants have a modular structure, consisting of a kinase domain and an acidic E and/or D rich domain. Therefore, it is envisaged that engineering of the kinase and/or acidic domains, in such a way that the activity of the SnRK2 protein is retained or modified, is useful in performing the methods of the invention. Preferred variants include those generated by domain deletion, stacking or shuffling (see for example He et al., Science 288, 2360-2363, 2000; or U.S. Pat. Nos. 5,811,238 and 6,395,547).

The term portion as defined herein refers to a piece of DNA comprising at least 700 nucleotides and which portion comprises a functional kinase domain, the conserved signature sequence of SEQ ID NO: 6 and an acidic C-terminal domain as defined above. A portion may be prepared, for example, by making one or more deletions to an SnRK2 nucleic acid. The portions may be used in isolated form or they may be fused to other coding (or non coding) sequences in order to, for example, produce a protein that combines several activities, one of them being protein kinase activity. When fused to other coding sequences, the resulting polypeptide produced upon translation may be bigger than that predicted for the SnRK2 fragment. Portions useful in the methods of the present invention comprise at least a functional kinase domain, the conserved signature sequence of SEQ ID NO: 6 and an acidic C-terminal domain as defined above. The functional portion may be a portion of a nucleic acids as represented by any one of SEQ ID NO: 7, 9, 11, 13, 15,17, 19, 21, 23, 25, 27, 29, 31, 33, 35, 37, 39, 41,43, 45, 47, 49, 51 and 53. Preferably, the functional portion is a portion of a nucleic acid as represented by SEQ ID NO: 1.

The term “hybridisation” as defined herein is a process wherein substantially homologous complementary nucleotide sequences anneal to each other. The hybridisation process may occur entirely in solution, i.e. both complementary nucleic acids are in solution. The hybridisation process may also occur with one of the complementary nucleic acids immobilised to a matrix such as magnetic beads, Sepharose beads or any other resin. The hybridisation process may furthermore occur with one of the complementary nucleic acids immobilised to a solid support such as a nitrocllulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids. The stringency of hybridisation is influenced by conditions such as temperature, salt concentration, ionic strength and hybridisation buffer composition.

“Stringent hybridisation conditions” and “stringent hybridisation wash conditions” in the context of nucleic acid hybridisation experiments such as Southern and Northern hybridisations are sequence dependent and are different under different environmental parameters. The skilled artisan is aware of various parameters which may be altered during hybridisation and washing and which will either maintain or change the stringency conditions.

The Tₘ is the temperature under defined ionic strength and pH, at which 50% of the target sequence hybridises to a perfectly matched probe. The Tₘ is dependent upon the solution conditions and the base composition and length of the probe. For example, longer sequences hybridise specifically at higher temperatures. The maximum rate of hybridisation is obtained from about 16° C. up to 32°C. below Tₘ. The presence of monovalent cations in the hybridisation solution reduce the electrostatic repulsion between the two nucleic acid strands thereby promoting hybrid formation; this effect is visible for sodium concentrations of up to 0.4M. Formamide reduces the malting temperature of DNA-DNA and DNA-RNA duplexes with 0.6 to 0.7° C. for each percent formamide, and addition of 50% formamide allows hybridisation to be performed at 30 to 45°C., though the rate of hybridisation will be lowered. Base pair mismatches reduce the hybridisation rate and the thermal stability of the duplexes. On average and for large probes, the Tₘ decreases about 1° C. per % base mismatch. The Tₘ may be calculated using the following equations, depending on the types of hybrids:
DNA-DNA hybrids (Meinkoth and Wahl, Anal. Biochem., 138: 267-284, 1984):
   Tₘ=81.5° C.+16.6xlog[Na⁺]^{a}+0.41x % [G/C^{b}]−500x[L^{c}]⁻¹−0.61x % formamide
DNA-RNA or RNA-RNA hybrids:
   Tₘ=79.8+18.5 (log₁₀[Na⁺]^{a})+0.58 (% G/C^{b})+11.8 (% G/C^{b})²−820/L^{c}
oligo-DNA or oligo-RNA^{d} hybrids:
   For <20 nucleotides: Tₘ=2 (Iₙ)
   For 20-35 nucleotides: Tₘ=22+1.46 (Iₙ)
      ^{a}or for other monovalent cation, but only accurate in the 0.01-0.4 M range.
      ^{b} only accurate for % GC in the 30% to 75% range.
      ^{c} L=length of duplex in base pairs.
      ^{d} Oligo, oligonucleotide; Iₙ, effective length of primer=2×(no. of G/C)+(no. of A/T).

Note: for each 1% formamide, the Tₘ is reduced by about 0.6 to 0.7° C., while the presence of 6M urea reduces the Tₘ by about 30° C.

Specificity of hybridisation is typically the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. Generally, suitable stringent conditions for nucleic acid hybridisation assays or gene amplification detection procedures are as set forth above. More or less stringent conditions may also be selected. Generally, low stringency conditions are selected to be about 50° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. For example, stringent conditions are those that are at least as stringent as, for example, conditions A-L; and reduced stringency conditions are at least as stringent as, for example, conditions M-R. Non-specific binding may be controlled using any one of a number of known techniques such as, for example, blocking the membrane with protein containing solutions, additions of heterologous RNA, DNA, and SDS to the hybridisation buffer, and treatment with Rnase.

Examples of hybridisation and wash conditions are listed in table 1:

TABLE 1

| | | | | Wash |
|---|---|---|---|---|
| Stringency | Polynucleotide | Hybrid Length | Hybridization Temperature | Temperature |
| Condition | Hybrid^{±} | (bp)^{‡} | and Buffer^{†} | and Buffer^{†} |
| A | DNA:DNA | > or | 65° C. 1xSSC; or 42° C., 1xSSC | 65° C.; 0.3xSSC |
| | | equal to 50 | and 50% formamide | |
| B | DNA:DNA | <50 | Tb*; 1xSSC | Tb*; 1xSSC |
| C | DNA:RNA | > or | 67° C. 1xSSC; or 45° C., 1xSSC | 67° C.; 0.3xSSC |
| | | equal to 50 | and 50% formamide | |
| D | DNA:RNA | <50 | Td*; 1xSSC | Td*; 1xSSC |
| E | RNA:RNA | > or | 70° C. 1xSSC; or 50° C., 1xSSC | 70° C.; 0.3xSSC |
| | | equal to 50 | and 50% formamide | |
| F | RNA:RNA | <50 | Tf*; 1xSSC | Tf*; 1xSSC |
| G | DNA:DNA | > or | 65° C. 4xSSC; or 45° C., 4xSSC | 65° C.; 1xSSC |
| | | equal to 50 | and 50% formamide | |
| H | DNA:DNA | <50 | Th*; 4xSSC | Th*; 4xSSC |
| I | DNA:RNA | > or | 67° C. 4xSSC; or 45° C., 4xSSC | 67° C.; 1xSSC |
| | | equal to 50 | and 50% formamide | |
| J | DNA:RNA | <50 | Tj*; 4xSSC | Tj*; 4 xSSC |
| K | RNA:RNA | > or | 70° C. 4xSSC; or 40° C., 6xSSC | 67° C.; 1xSSC |
| | | equal to 50 | and 50% formamide | |
| L | RNA:RNA | <50 | Tl*; 2xSSC | Tl*; 2xSSC |
| M | DNA:DNA | > or | 50° C. 4xSSC; or 40° C., 6xSSC | 50° C.; 2xSSC |
| | | equal to 50 | and 50% formamide | |
| N | DNA:DNA | <50 | Tn*; 6xSSC | Tn*; 6xSSC |
| O | DNA:RNA | > or | 55° C. 4xSSC; or 42° C., 6xSSC | 55° C.; 2xSSC |
| | | equal to 50 | and 50% formamide | |
| P | DNA:RNA | <50 | Tp*; 6xSSC | Tp*; 6xSSC |
| Q | RNA:RNA | > or | 60° C. 4xSSC; or 45° C., 6xSSC | 60° C.; 2xSSC |
| | | equal to 50 | and 50% formamide | |
| R | RNA:RNA | <50 | Tr*; 4xSSC | Tr*; 4xSSC |

^{‡}The “hybrid length” is the anticipated length for the hybridising nucleic acid. When nucleic acids of known sequence are hybridised, the hybrid length may be determined by aligning the sequences and identifying the conserved regions described herein.
^{†}SSPE (1xSSPE is 0.15M NaCl, 10 mM NaH₂PO₄, and 1.25 mM EDTA, pH 7.4) may be substituted for SSC (1xSSC is 0.15M NaCl and 15 mM sodium citrate) in the hybridisation and wash buffers; washes are performed for 15 minutes after hybridisation is complete. The hybridisations and washes may additionally include 5 × Denhardt's reagent, 0.5-1.0% SDS, 100 μg/ml denatured, fragmented salmon sperm DNA, 0.5% sodium pyrophosphate, and up to 50% formamide.
*Tb-Tr: The hybridisation temperature for hybrids anticipated to be less than 50 base pairs in length should be 5-10° C. less than the melting temperature Tₘ of the hybrids; the Tₘ is determined according to the above-mentioned equations.
^{±}The present invention also encompasses the substitution of any one, or more DNA or RNA hybrid partners with either a PNA, or a modified nucleic acid.

For the purposes of defining the level of stringency, reference may conveniently be made to Sambrook et al. (2001) Molecular Cloning: a laboratory manual, 3^{rd} Edition Cold Spring Harbor Laboratory Press, CSH, New York or to Current Protocols in Molecular Biology, John Wiley & Sons, N.Y. (1989).

For example, a nucleic acid encoding SEQ ID NO: 2 or a homologue thereof may be used in a hybridisation experiment. Alternatively fragments thereof may be used as probes. Depending on the starting pool of sequences from which the SnRK2 protein is to be identified, different fragments for hybridization may be selected. For example, when a limited number of homologues with a high sequence identity to SnRK2 are desired, a less conserved fragment may be used for hybridisation. By aligning SEQ ID NO: 2 and homologues thereof, it is possible to design equivalent nucleic acid fragments useful as probes for hybridisation.

After hybridisation and washing, the duplexes may be detected by autoradiography (when radiolabeled probes were used) or by chemiluminescence, immunodetection, by fluorescent or chromogenic detection, depending on the type of probe labelling. Alternatively, a ribonuclease protection assay may be performed for detection of RNA:RNA hybrids.

The SnRK2 nucleic acid molecule or functional variant thereof may be derived from any natural or artificial source. The nucleic acid/gene or functional variant thereof may be isolated from a microbial source, such as bacteria, yeast or fungi, or from a plant, alga or animal (including human) source. This nucleic acid may be modified from its native form in composition and/or genomic environment through deliberate human manipulation. The nucleic acid is preferably of plant origin, whether from the same plant species (for example to the one in which it is to be introduced) or whether from a different plant species. The nucleic acid may be isolated from a dicotyledonous species, preferably from the family Brassicaceae, further preferably from Arabidopsis thaliana. More preferably, the SnRK2 isolated from Arabidopsis thaliana is represented by SEQ ID NO: 1 and the SnRK2 amino acid sequence is as represented by SEQ ID NO: 2.

The SnRK2 polypeptide or homologue thereof may be encoded by an alternative splice variant of an SnRK2 nucleic acid molecule or gene. The term “alternative splice variant” as used herein encompasses variants of a nucleic acid sequence in which selected introns and/or exons have been excised, replaced or added. Such variants will be ones in which the biological activity of the protein as outlined above is retained, which may be achieved by selectively retaining functional segments of the protein. Such splice variants may be found in nature or may be manmade. Methods for making such splice variants are well known in the art. Preferred splice variants are all splice variants derived from the nucleic acid represented by SEQ ID NO: 3, such as SEQ ID NO: 1. Further preferred are splice variants encoding a polypeptide having a functional kinase domain flanked by the conserved signature sequence of SEQ ID NO: 6 and the C-terminal acidic domain defined above.

The homologue may also be encoded by an allelic variant of a nucleic acid encoding an SnRK2 polypeptide or a homologue thereof, preferably an allelic variant of the nucleic acid represented by SEQ ID NO: 1. Further preferably, the polypeptide encoded by the allelic variant has a functional kinase domain flanked by the conserved signature sequence of SEQ ID NO: 6 and the C-terminal acidic domain defined above. Allelic variants exist in nature and encompassed within the methods of the present invention is the use of these natural alleles. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

The activity and/or expression of an SnRK2 polypeptide or a homologue thereof may be increased by introducing a genetic modification (preferably in the locus of an SnRK2 gene). The locus of a gene as defined herein is taken to mean a genomic region which includes the gene of interest and 10 kb up or downstream of the coding region.

The genetic modification may be introduced, for example, by any one (or more) of the following methods: TDNA activation, TILLING, site-directed mutagenesis, homologous recombination, directed evolution or by introducing and expressing in a plant a nucleic acid encoding an SnRK2 polypeptide or a homologue thereof. Following introduction of the genetic modification there follows a step of selecting for increased activity and/or expression of an SnRK2 polypeptide, which increase in activity and/or expression gives plants having improved growth characteristics.

T-DNA activation tagging (Hayashi et al. Science 258, 1350-1353, 1992) involves insertion of T-DNA usually containing a promoter (may also be a translation enhancer or an intron), in the genomic region of the gene of interest or 10 KB up- or down stream of the coding region of a gene in a configuration such that such promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to overexpression of genes near to the inserted T-DNA. The resulting transgenic plants show dominant phenotypes due to overexpression of genes dose to the introduced promoter. The promoter to be introduced may be any promoter capable of directing expression of a gene in the desired organism, in this case a plant. For example, constitutive, tissue-preferred, cell type-preferred and inducible promoters are all suitable for use in T-DNA activation.

A genetic modification may also be introduced in the locus of an SnRK2 gene using the technique of TILLING (Targeted Induced Local Lesions IN Genomes). This is a mutagenesis technology useful to generate and/or identify, and to isolate mutagenised variants of an SnRK2 nucleic acid molecule capable of exhibiting SnRK2 activity. TILLING also allows selection of plants carrying such mutant variants. These mutant variants may even exhibit higher SnRK2 activity than that exhibited by the gene in its natural form. TILLING combines high-density mutagenesis with high-throughput screening methods. The steps typically followed in TILLING are: (a) EMS mutagenesis (Redei and Koncz (1992), In: C Koncz, N-H Chua, J Schell, eds, Methods in Arabidopsis Research. World Scientific, Singapore, pp 1682; Feldmann et al., (1994) In: E M Meyerowitz, C R Somerville, eds, Arabidopsis. Cold Spring Harbor Laboratory Press, Cold Spring Harbor, N.Y., pp 137-172; Lightner and Caspar (1998), In: J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum Nature Biotechnol. 18, 455-457, 2000, Stemple Nature Rev. Genet. 5, 145-150, 2004).

Site directed mutagenesis may be used to generate variants of SnRK2 nucleic acids or portions thereof that retain activity, namely, protein kinase activity. Several methods are available to achieve site directed mutagenesis, the most common being PCR based methods (See for example Ausubal et al., Current Protocols in Molecular Biology. Wiley Eds. http://www.4ulr.com/products/currentprotocols/index.html).

Directed evolution may be used to generate functional variants of SnRK2 nucleic acid molecules encoding SnRK2 polypeptides or homologues, or portions thereof having an increased biological activity as outlined above. Directed evolution consists of iterations of DNA shuffling followed by appropriate screening and/or selection (Castle et al., (2004) Science 304(5674): 1151-4; U.S. Pat. Nos. 5,811,238 and 6,395,547).

TDNA activation, TILLING, site-directed mutagenesis and directed evolution are examples of technologies that enable the generation novel alleles and functional variants of SnRK2 that retain SnRK2 function as outlined above and which are therefore useful in the methods of the invention.

Homologous recombination allows introduction in a genome of a selected nucleic acid at a defined selected position. Homologous recombination is a standard technology used routinely in biological sciences for lower organism such as yeast or the moss Physcomitrella. Methods for performing homologous recombination in plants have been described not only for model plants (Offringa et al. (1990) EMBO J. 9, 3077-3084) but also for crop plants, for example rice (Terada et al., (2002) Nature Biotechnol. 20, 1030-1034; or lida and Terada (2004) Curr. Opin. Biotechnol. 15, 132-138). The nucleic acid to be targeted (which may be an SnRK2 nucleic acid molecule or functional variant thereof as hereinbefore defined) need not be targeted to the locus of an SnRK2 gene, but may be introduced in, for example, regions of high expression. The nucleic acid to be targeted may be an improved allele used to replace the endogenous gene or may be introduced in addition to the endogenous gene.

According to a preferred embodiment of the invention, plant growth characteristics may be improved by introducing and expressing in a plant a nucleic acid encoding an SnRK2 polypeptide or a homologue thereof.

A preferred method for introducing a genetic modification (which in this case need not be in the locus of an SnRK2 gene) is to introduce and express in a plant a nucleic acid encoding an SnRK2 polypeptide or a homologue thereof. An SnRK2 polypeptide or a homologue thereof as mentioned above is one having kinase activity and, in increasing order of preference, having at least 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid sequence represented by SEQ ID NO: 2, and furthermore comprising a kinase domain, the conserved signature sequence as represented by SEQ ID NO: 6 and a C-terminal acidic domain as defined above.

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A homologue may be in the form of a “substitutional variant” of a protein, i.e. where at least one residue in an amino add sequence has been removed and a different residue inserted in its place. Amino add substitutions are typically of single residues, but may be clustered depending upon functional constraints placed upon the polypeptide; insertions will usually be of the order of about 1 to 10 amino add residues. Preferably, amino add substitutions comprise conservative amino add substitutions (Table 2). To produce such homologues, amino acids of the protein may be replaced by other amino acids having similar properties (such as similar hydrophobicity, hydrophilicity, antigenicity, propensity to form or break α-helical structures or β-sheet structures). Conservative substitution tables are well known in the art (see for example Creighton (1984) Proteins. W.H. Freeman and Company).

TABLE 2
Examples of conserved amino acid substitutions:

| | Conservative | | Conservative |
|---|---|---|---|
| Residue | Substitutions | Residue | Substitutions |
| Ala | Ser | Leu | Ile; Val |
| Arg | Lys | Lys | Arg; Gln |
| Asn | Gln; His | Met | Leu; Ile |
| Asp | Glu | Phe | Met; Leu; Tyr |
| Gln | Asn | Ser | Thr; Gly |
| Cys | Ser | Thr | Ser; Val |
| Glu | Asp | Trp | Tyr |
| Gly | Pro | Tyr | Trp; Phe |
| His | Asn; Gln | Val | Ile; Leu |
| Ile | Leu, Val | | |

Less conserved substitutions may be made in case the above-mentioned amino acid properties are not so critical.

A homologue may also be in the form of an “insertional variant” of a protein, i.e. where one or more amino acid residues are introduced into a predetermined site in a protein. Insertions may comprise amino-terminal and/or carboxy-terminal fusions as well as intra-sequence insertions of single or multiple amino acids. Generally, insertions within the amino acid sequence will be smaller than amino- or carboxy-terminal fusions, of the order of about 1 to 10 residues. Examples of amino- or carboxy-terminal fusion proteins or peptides include the binding domain or activation domain of a transcriptional activator as used in the yeast two-hybrid system, phage coat proteins, (histidine) 6-tag, glutathione S-transferase-tag, protein A, maltose-binding protein, dihydrofolate reductase, Tag 100 epitope, c-myc epitope, FLAG®-epitope, lacZ, CMP (calmodulin-binding peptide), HA epitope, protein C epitope and VSV epitopa.

Homologues in the form of “deletion variants” of a protein are characterised by the removal of one or more amino acids from a protein.

Amino acid variants of a protein may readily be made using peptide synthetic techniques well known in the art, such as solid phase peptide synthesis and the like, or by recombinant DNA manipulations. Methods for the manipulation of DNA sequences to produce substitution, insertion or deletion variants of a protein are well known in the art. For example, techniques for making mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

The SnRK2 polypeptide or homologue thereof may be a derivative. “Derivative” include peptides, oligopeptides, polypeptides, proteins and enzymes which may comprise substitutions, deletions or additions of naturally and non-naturally occurring amino acid residues compared to the amino add sequence of a naturally-occurring form of the protein, for example, as presented in SEQ ID NO: 2. “Derivatives” of a protein encompass peptides, oligopeptides, polypeptides, proteins and enzymes which may comprise naturally occurring altered, glycosylated, acylated or non-naturally occurring amino acid residues compared to the amino acid sequence of a naturally-occurring form of the polypeptide. A derivative may also comprise one or more non-amino acid substituents compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein.

According to a preferred aspect of the present invention, enhanced or increased expression of the SnRK2 nucleic acid molecule or functional variant thereof is envisaged. Methods for obtaining enhanced or increased expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of an SnRK2 nucleic acid or functional variant thereof. For example, endogenous promoters may be altered in vivo by mutation, deletion, and/or substitution (see, Kmiec, U.S. Pat. No. 5,565,350; Zarling et al., PCT/US93/03868), or isolated promoters may be introduced into a plant cell in the proper orientation and distance from a gene of the present invention so as to control the expression of the gene.

If polypeptide expression is desired, it is generally desirable to include a polyadenylation region at the 3′-end of a polynucleotide coding region. The polyadenylation region may be derived from a natural gene, from a variety of other plant genes, or from T-DNA. The 3′ end sequence to be added may be derived from, for example, the nopaline synthase or octopine synthase genes, or alternatively from other plant genes, or less preferably from any other eukaryotic gene.

An intron sequence may also be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, Mol. Cell Biol. 8, 4395-4405 (1988); Callis et al., Genes Dev. 1, 1183-1200 (1987)). Such intron enhancement of gene expression is typically greatest when placed near the 5′ end of the transcription unit. Use of the maize introns Adh1-S intron 1, 2, and 6, the Bronze-1 intron are known in the art. See generally, The Maize Handbook, Chapter 116, Freeling and Walbot, Eds., Springer, N.Y. (1994).

The invention also provides genetic constructs and vectors to facilitate introduction and/or expression of the nucleotide sequences useful in the methods according to the invention.

Therefore, there is provided a gene construct comprising:
(i) an SnRK2 nucleic acid molecule or functional variant thereof;
(ii) one or more control sequence capable of driving expression of the nucleic acid sequence of (i); and optionally
(iii) a transcription termination sequence.

Constructs useful in the methods according to the present invention may be constructed using recombinant DNA technology well known to persons skilled in the art. The gene constructs may be inserted into vectors, which may be commercially available, suitable for transforming into plants and suitable for expression of the gene of interest in the transformed cells.

Plants are transformed with a vector comprising the sequence of interest (i.e., an SnRK2 nucleic acid or functional variant thereof). The sequence of interest is operably linked to one or more control sequences (at least to a promoter). The terms “regulatory element”, “control sequence” and “promoter” are all used interchangeably herein and are to be taken in a broad context to refer to regulatory nucleic acid sequences capable of effecting expression of the sequences to which they are ligated. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative which confers, activates or enhances expression of a nucleic acid molecule in a call, tissue or organ. The term “operably linked” as used herein refers to a functional linkage between the promoter sequence and the gene of interest, such that the promoter sequence is able to initiate transcription of the gene of interest.

Advantageously, any type of promoter may be used to drive expression of the nucleic acid sequence. The promoter may be an inducible promoter, i.e. having induced or increased transcription initiation in response to a developmental, chemical, environmental or physical stimulus. An example of an inducible promoter being a stress-inducible promoter, i.e. a promoter activated when a plant is exposed to various stress conditions, is the water stress induced promoter WSI18. Additionally or alternatively, the promoter may be a tissue-specific promoter, i.e. one that is capable of preferentially initiating transcription in certain tissues, such as the leaves, roots, seed tissue etc. An example of a seed-specific promoter is the rice oleosin 18 kDa promoter (Wu et al. (1998) J Biochem 123(3): 386-91).

Preferably, the SnRK2 nucleic add or functional variant thereof is operably linked to a constitutive promoter. The term “constitutive” as defined herein refers to a promoter that is expressed predominantly in at least one tissue or organ and predominantly at any life stage of the plant. Preferably the promoter is expressed predominantly throughout the plant. Preferably, the constitutive promoter capable of preferentially expressing the nucleic acid throughout the plant has a comparable expression profile to a GOS2 promoter. More preferably, the constitutive promoter has the same expression profile as the rice GOS2 promoter, most preferably, the promoter capable of preferentially expressing the nucleic acid throughout the plant is the GOS2 promoter from rice represented in SEQ ID NO: 55. It should be dear that the applicability of the present invention is not restricted to the SnRK2 nucleic acid represented by SEQ ID NO: 1, nor is the applicability of the invention restricted to expression of an SnRK2 nucleic acid when driven by a GOS2 promoter. Examples of other constitutive promoters that may also be used to drive expression of a SnRK2 nucleic acid are shown in Table 3 below.

TABLE 3
Examples of constitutive promoters

| | Expression | |
|---|---|---|
| Gene Source | Motif | Reference |
| Actin | Constitutive | McElroy et al, Plant Cell, 2: 163-171, |
| | | 1990 |
| CAMV 35S | Constitutive | Odell et al, Nature, 313: 810-812, |
| | | 1985 |
| CaMV 19S | Constitutive | Nilsson et al., Physiol. Plant. |
| | | 100: 456-462, 1997 |
| GOS2 | Constitutive | de Pater et al, Plant J Nov; 2(6): |
| | | 837-44, 1992 |
| Ubiquitin | Constitutive | Christensen et al, Plant Mol. Biol. |
| | | 18: 675-689, 1992 |
| Rice cyclophilin | Constitutive | Buchholz et al, Plant Mol Biol. 25(5): |
| | | 837-43, 1994 |
| Maize H3 histone | Constitutive | Lepetit et al, Mol. Gen. Genet. |
| | | 231: 276-285, 1992 |
| Actin 2 | Constitutive | An et al, Plant J. 10(1); 107-121, |
| | | 1996 |

Optionally, one or more terminator sequences may also be used in the construct introduced into a plant. The term “terminator” encompasses a control sequence which is a DNA sequence at the end of a transcriptional unit which signals 3′ processing and polyadenylation of a primary transcript and termination of transcription. Additional regulatory elements may include transcriptional as well as translational enhancers. Those skilled in the art will be aware of terminator and enhancer sequences which may be suitable for use in performing the invention. Such sequences would be known or may readily be obtained by a person skilled in the art.

The genetic constructs of the invention may further include an origin of replication sequence, which is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

The genetic construct may optionally comprise a selectable marker gene. As used herein, the term “selectable marker gene” includes any gene which confers a phenotype on a cell in which it is expressed to facilitate the identification and/or selection of cells which are transfected or transformed with a nucleic acid construct of the invention. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin), to herbicides (for example bar which provides resistance to Basta; aroA or gox providing resistance against glyphosate), or genes that provide a metabolic trait (such as manA that allows plants to use mannose as sole carbon source). Visual marker genes result in the formation of colour (for example β-glucuronidase, GUS), luminescence (such as luciferase) or fluorescence (Green Fluorescent Protein, GFP, and derivatives thereof).

The present invention also encompasses plants obtainable by the methods according to the present invention. The present invention therefore provides plants obtainable by the method according to the present invention, which plants have introduced therein an SnRK2 nucleic acid or functional variant thereof, or which plants have introduced therein a genetic modification, preferably in the locus of an SnRK2 gene.

The invention also provides a method for the production of transgenic plants having improved growth characteristics, comprising introduction and expression in a plant of an SnRK2 nucleic acid or a functional variant thereof.

More specifically, the present invention provides a method for the production of transgenic plants having improved growth characteristics, which method comprises:
(i) introducing into a plant or plant cell an SnRK2 nucleic acid or functional variant thereof; and
(ii) cultivating the plant cell under conditions promoting plant growth and development.

The nucleic acid may be introduced directly into a plant cell or into the plant itself (including introduction into a tissue, organ or any other part of a plant). According to a preferred feature of the present invention, the nucleic acid is preferably introduced into a plant by transformation.

The term “transformation” as referred to herein encompasses the transfer of an exogenous polynudeotide into a host cell, irrespective of the method used for transfer. Plant tissue capable of subsequent clonal propagation, whether by organogenesis or embryogenesis, may be transformed with a genetic construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant call may then be used to regenerate a transformed plant in a manner known to persons skilled in the art.

Transformation of plant species is now a fairly routine technique. Advantageously, any of several transformation methods may be used to introduce the gene of interest into a suitable ancestor cell. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens et al. (1982) Nature 296, 72-74; Negrutiu et al. (1987) Plant Mol. Biol. 8, 363-373); electroporation of protoplasts (Shillito et al. (1985) Bio/Technol 3, 1099-1102); microinjection into plant material (Crossway et al. (1986) Mol. Gen. Genet. 202, 179-185); DNA or RNA-coated particle bombardment (Klein et al. (1987) Nature 327, 70) infection with (non-integrative) viruses and the like. Transgenic rice plants expressing an SnRK2 transgene are preferably produced via Agrobacterium-mediated transformation using any of the well known methods for rice transformation, such as described in any of the following: published European patent application EP 1198985 A1, Aldemita and Hodges (Planta 199, 612-617, 1996); Chan et al. (Plant Mol. Biol. 22, 491-506, 1993), Hiei et al. (Plant J. 6, 271-282, 1994), which disclosures are incorporated by reference herein as if fully set forth. In the case of com transformation, the preferred method is as described in either Ishida et al. (Nature Biotechnol. 14, 745-50, 1996) or Frame et al. (Plant Physiol. 129, 13-22, 2002), which disclosures are incorporated by reference herein as if fully set forth.

Generally after transformation, plant cells or call groupings are selected for the presence of one or more markers which are encoded by plant-expressible genes co-transferred with the gene of interest, following which the transformed material is regenerated into a whole plant.

Following DNA transfer and regeneration, putatively transformed plants may be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art. The cultivation of transformed plant cells into mature plants may thus encompass steps of selection and/or regeneration and/or growing to maturity.

The generated transformed plants may be propagated by a variety of means, such as by clonal propagation or classical breeding techniques. For example, a first generation (or T1) transformed plant may be selfed to give homozygous second generation (or T2) transformants, and the T2 plants further propagated through classical breeding techniques.

The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

The present invention dearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced in the parent by the methods according to the invention. The invention also includes host calls containing an isolated SnRK2 nucleic acid or functional variant thereof. Preferred host cells according to the invention are plant cells. The invention also extends to harvestable parts of a plant according to the invention such as but not limited to seeds, leaves, fruits, flowers, stems, rhizomes, tubers and bulbs. The invention furthermore relates to products directly derived from a harvestable part of such a plant, such as dry pellets or powders, oil, fat and fatty acids, starch or proteins.

The present invention also encompasses the use of SnRK2 nucleic acids or functional variants thereof and to the use of SnRK2 polypeptides or homologues thereof.

One such use relates to improving the growth characteristics of plants, in particular in improving yield, such as increased biomass and/or increased seed yield. The seed yield may include one or more of the following: increased number of (filled) seeds, increased seed weight, increased harvest index, increased thousand kernel weight, among others.

SnRK2 nucleic acids or variants thereof or SnRK2 polypeptides or homologues thereof may find use in breeding programmes in which a DNA marker is identified which may be genetically linked to an SnRK2 gene or variant thereof. The SnRK2 or variants thereof or SnRK2 proteins or homologues thereof may be used to define a molecular marker. This DNA or protein marker may then be used in breeding programs to select plants having improved growth characteristics. The SnRK2 gene or variant thereof may, for example, be a nucleic add as represented by SEQ ID NO: 1, or a nucleic acid encoding any of the above mentioned homologues.

Allelic variants of an SnRK2 gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place by, for example, PCR. This is followed by a selection step for selection of superior allelic variants of the sequence in question and which give improved growth characteristics in a plant. Selection is typically carried out by monitoring growth performance of plants containing different allelic variants of the sequence in question, for example, different allelic variants of SEQ ID NO: 1, or of nucleic acids encoding any of the above mentioned homologues. Growth performance may be monitored in a greenhouse or in the field. Further optional steps include crossing plants, in which the superior allelic variant was identified, with another plant. This could be used, for example, to make a combination of interesting phenotypic features.

An SnRK2 nucleic acid or variant thereof may also be used as a probe for genetically and physically mapping the genes that they are a part of, and as markers for traits linked to those genes. Such information may be useful in plant breeding in order to develop lines with desired phenotypes. Such use of SnRK2 nucleic acids or variants thereof requires only a nucleic acid sequence of at least 10 nucleotides in length. The SnRK2 nucleic adds or variants thereof may be used as restriction fragment length polymorphism (RFLP) markers. Southern blots of restriction-digested plant genomic DNA may be probed with the SnRK2 nucleic acids or variants thereof. The resulting banding patterns may then be subjected to genetic analyses using computer programs such as MapMaker (Lander et al. (1987) Genomics 1, 174-181) in order to construct a genetic map. In addition, the nucleic acids may be used to probe Southern blots containing restriction endonudease-treated genomic DNAs of a set of individuals representing parent and progeny of a defined genetic cross. Segregation of the DNA polymorphisms is noted and used to calculate the position of the SnRK2 nucleic acid or variant thereof in the genetic map previously obtained using this population (Botstein et al. (1980) Am. J. Hum. Genet. 32, 314-331).

The production and use of plant gene-derived probes for use in genetic mapping is described in Bernatzky and Tanksley (Plant Mol. Biol. Reporter 4, 37-41, 1986). Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Nonmammalian Genomic Analysis: A Practical Guide, Academic press 1996, pp. 319-346, and references cited therein).

In another embodiment, the nucleic acid probes may be used in direct fluorescence in situ hybridization (FISH) mapping (Trask (1991) Trends Genet. 7, 149-154). Although current methods of FISH mapping favour use of large clones (several to several hundred kb; see Laan et al. (1995) Genome Res. 5, 13-20), improvements in sensitivity may allow performance of FISH mapping using shorter probes.

A variety of nucleic acid amplification-based methods of genetic and physical mapping may be carried out using the nucleic acids. Examples include allele-specific amplification (Kazazian (1989) J. Lab. Clin. Med. 11, 9596), polymorphism of PCR-amplified fragments (CAPS; Sheffield et al. (1993) Genomics 16, 325-332), allele-specific ligation (Landegren et al. (1988) Science 241, 1077-1080), nucleotide extension reactions (Sokolov (1990) Nucleic Acid Res. 18, 3671), Radiation Hybrid Mapping (Walter et al. (1997) Nat. Genet. 7, 22-28) and Happy Mapping (Dear and Cook (1989) Nucleic Acid Res. 17, 6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic add sequence. This, however, is generally not necessary for mapping methods.

In this way, generation, identification and/or isolation of improved plants with altered SnRK2 activity and/or expression, displaying improved growth characteristics may be performed.

SnRK2 nucleic acids or functional variants thereof or SnRK2 polypeptides or homologues thereof may also find use as growth regulators. Since these molecules have been shown to be useful in improving the growth characteristics of plants, they would also be useful growth regulators, such as herbicides or growth stimulators. The present invention therefore provides a composition comprising an SnRK2 or functional variant thereof or an SnRK2 polypeptide or homologue thereof, together with a suitable carrier, diluent or excipient, for use as a growth regulator.

The methods according to the present invention result in plants having improved growth characteristics, as described hereinbefore. These advantageous growth characteristics may also be combined with other economically advantageous traits, such as further yield-enhancing traits, tolerance to various stresses, traits modifying various architectural features and/or biochemical and/or physiological features.

### DESCRIPTION OF FIGURES

The present invention will now be described with reference to the following figures in which:

FIG. 1 gives a graphical overview of SnRK2. The pentagram represents the kinase domain whereas the C-terminal region in light grey represents the Asp and/or Glu rich acidic region.

FIG. 2 shows a binary vector for transformation and expression in Oryza sativa of an Arabidopsis thaliana SnRK2 (internal reference CDS0758) under the control of a rice GOS2 promoter (internal reference PRO0129).

FIG. 3 details examples of sequences useful in performing the methods according to the present invention. SEQ ID NO: 1 and SEQ ID NO: 2 represent the nucleotide and protein sequence of SnRK2 used in the examples. SEQ ID NO: 3 represents the unspliced DNA sequence of SnRK2. SEQ ID NO: 4 and SEQ ID NO: 5 are primer sequences used for isolating the SnRK2 nucleic acid. SEQ ID NO: 6 represents a consensus sequence of a conserved part in the SnRK2 proteins. SEQ ID NO: 7 to 53 are nucleotide and protein sequences of homologues of the SnRK2 coding sequence and protein sequence as given in SEQ ID NO: 1 and SEQ ID NO: 2.

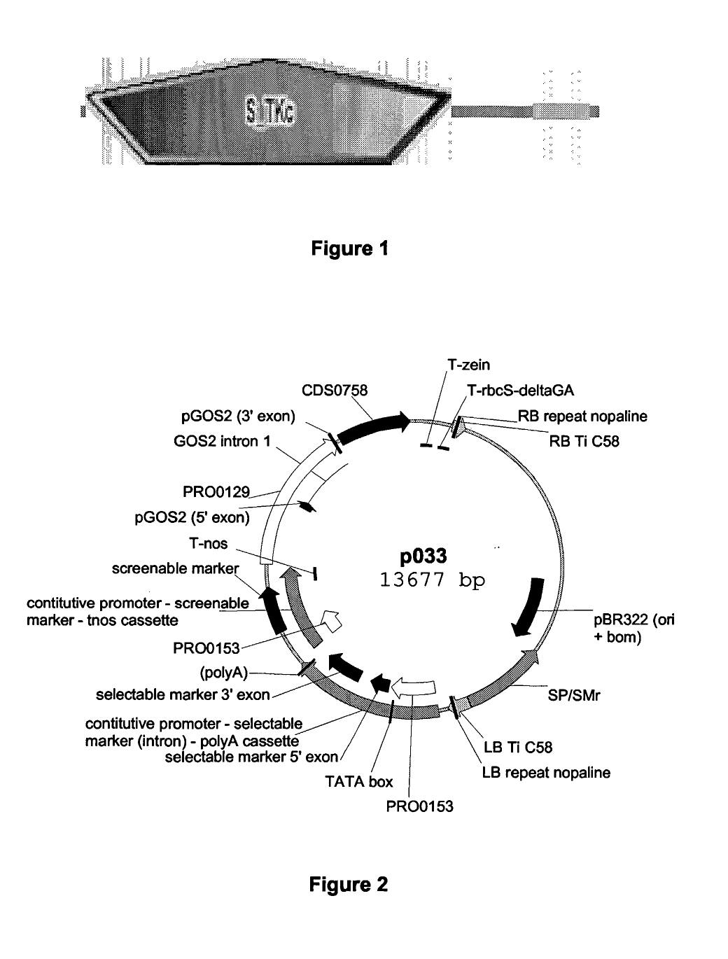

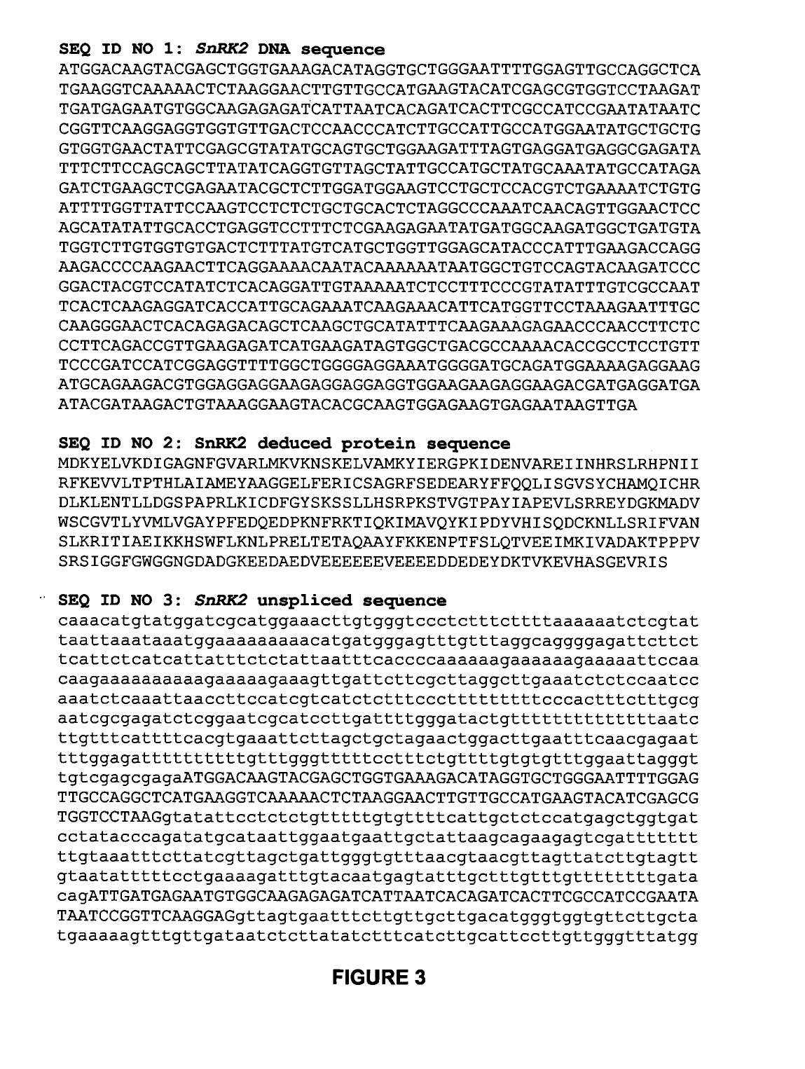

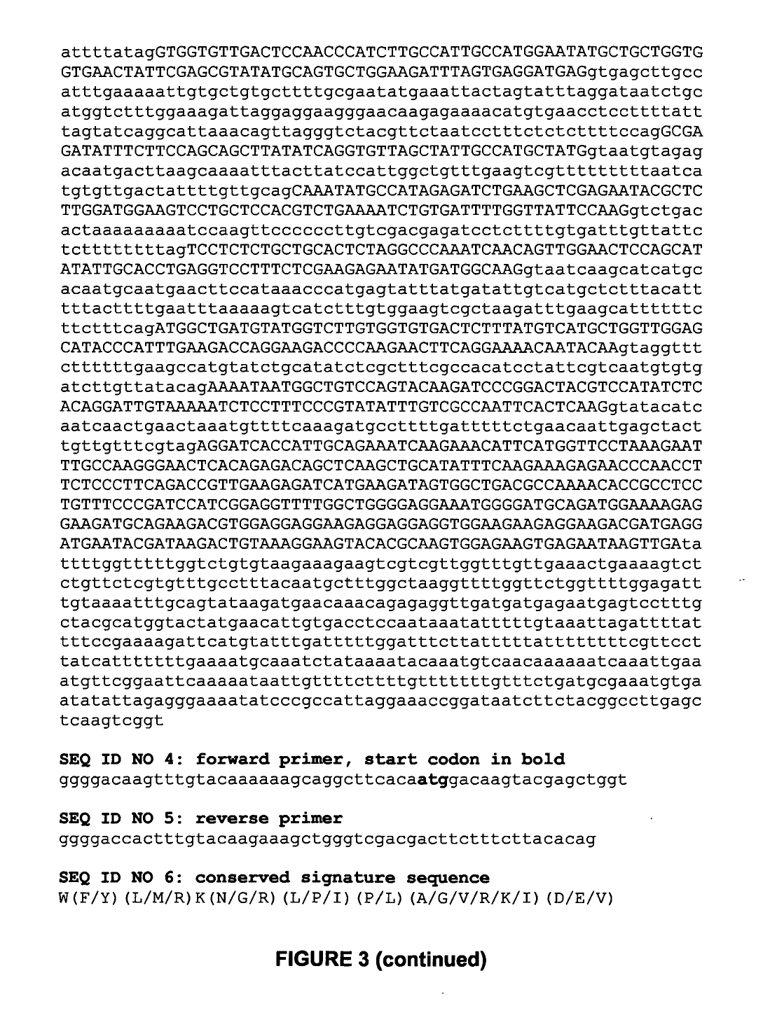

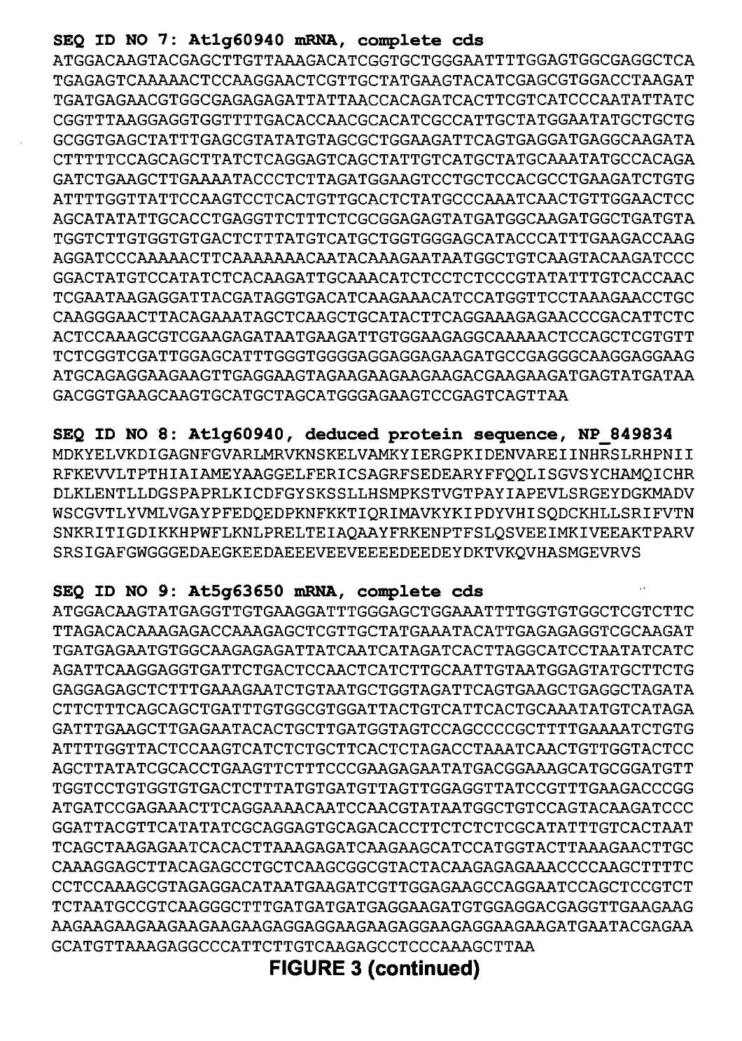

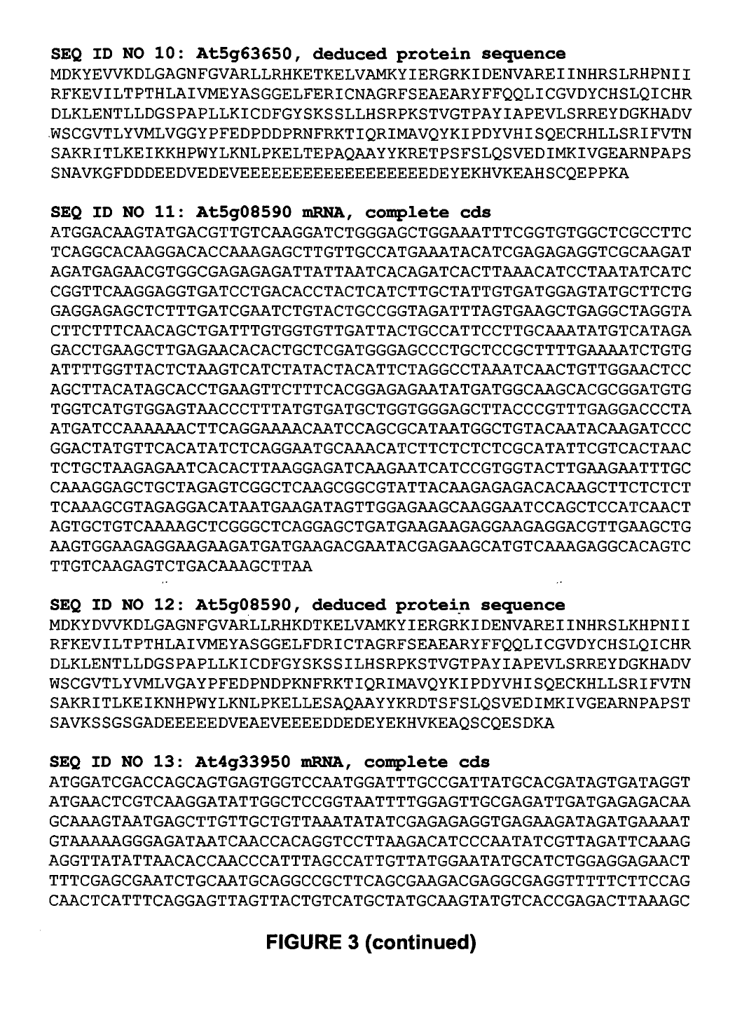

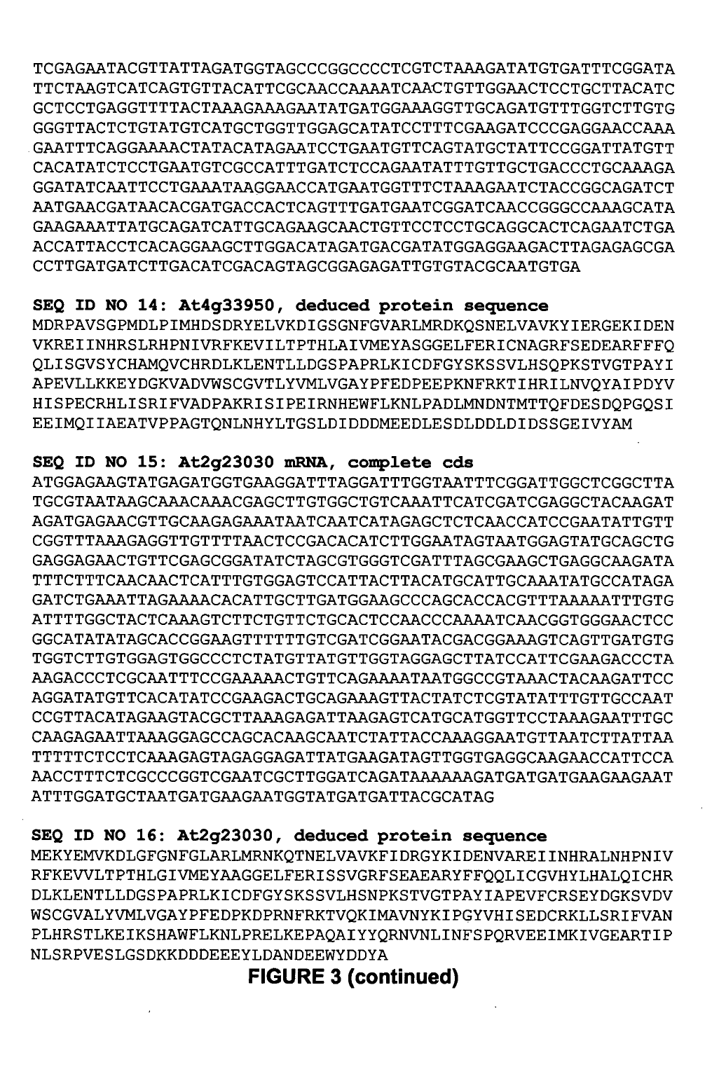

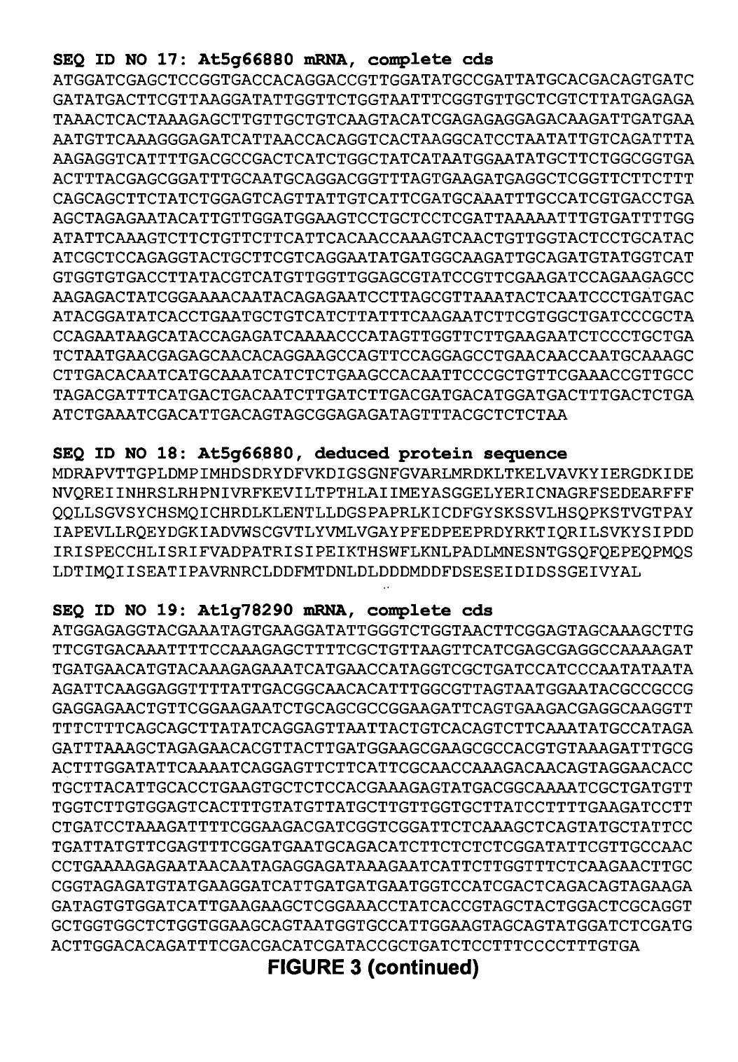

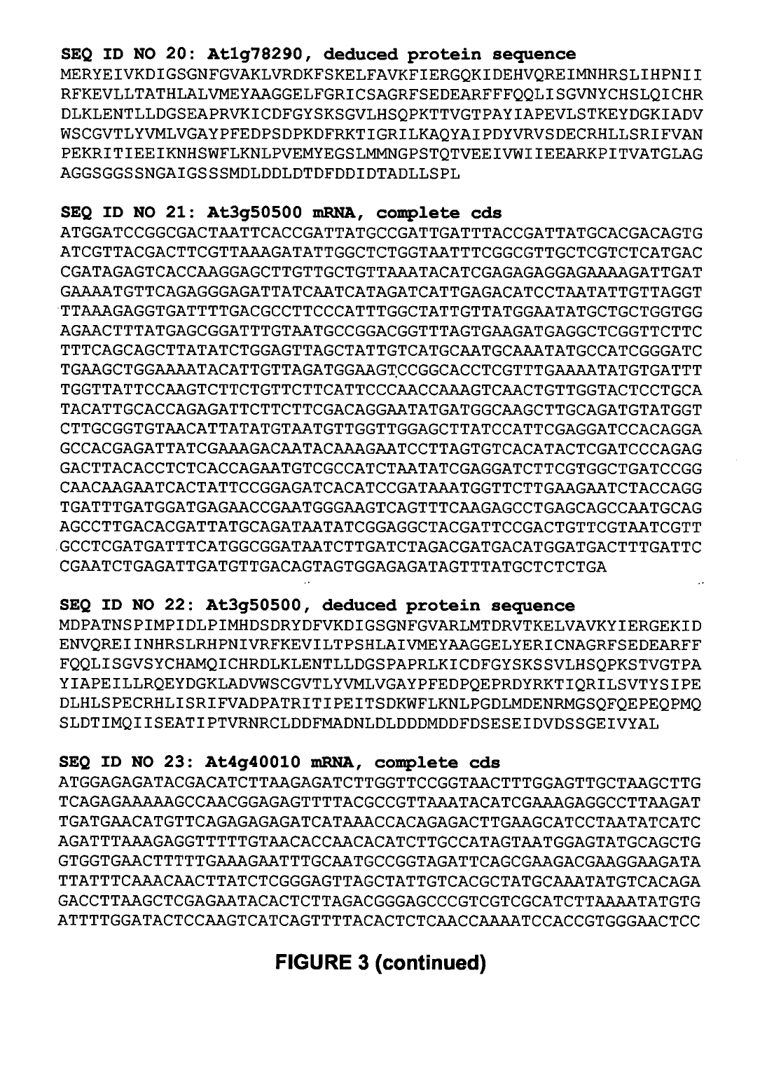

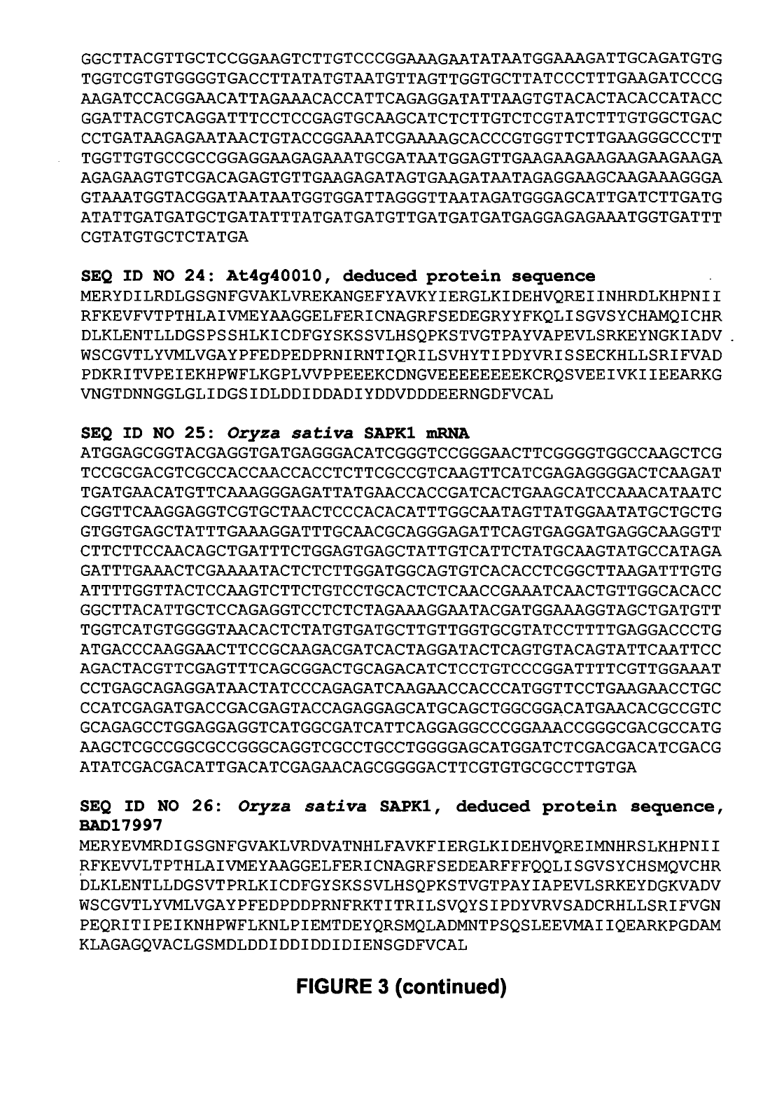

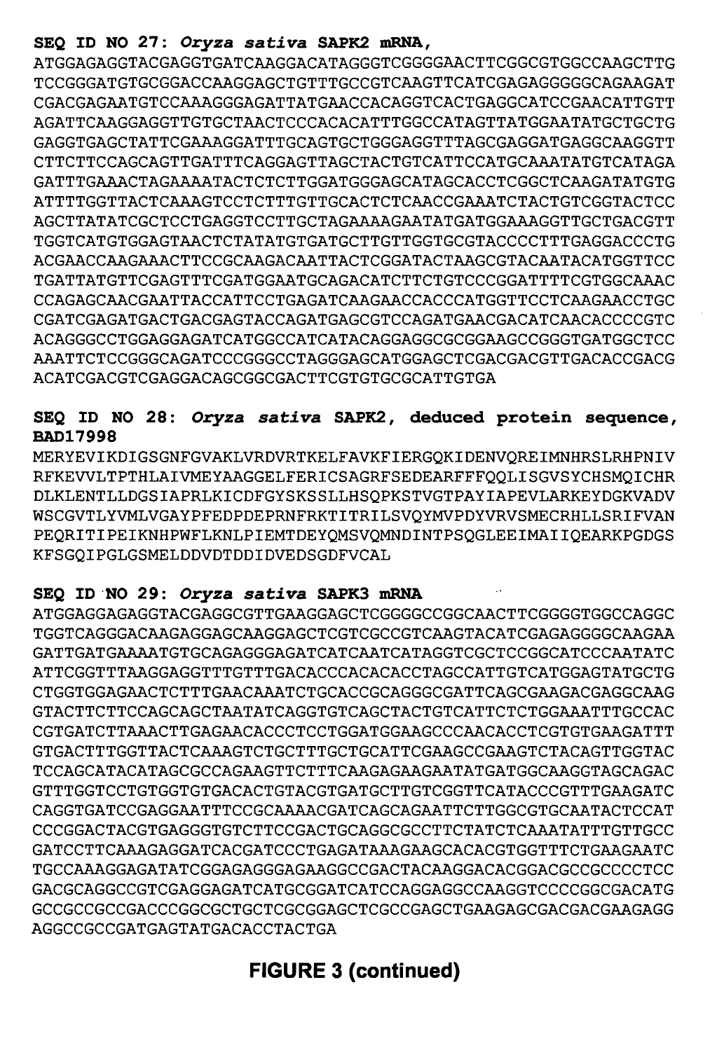

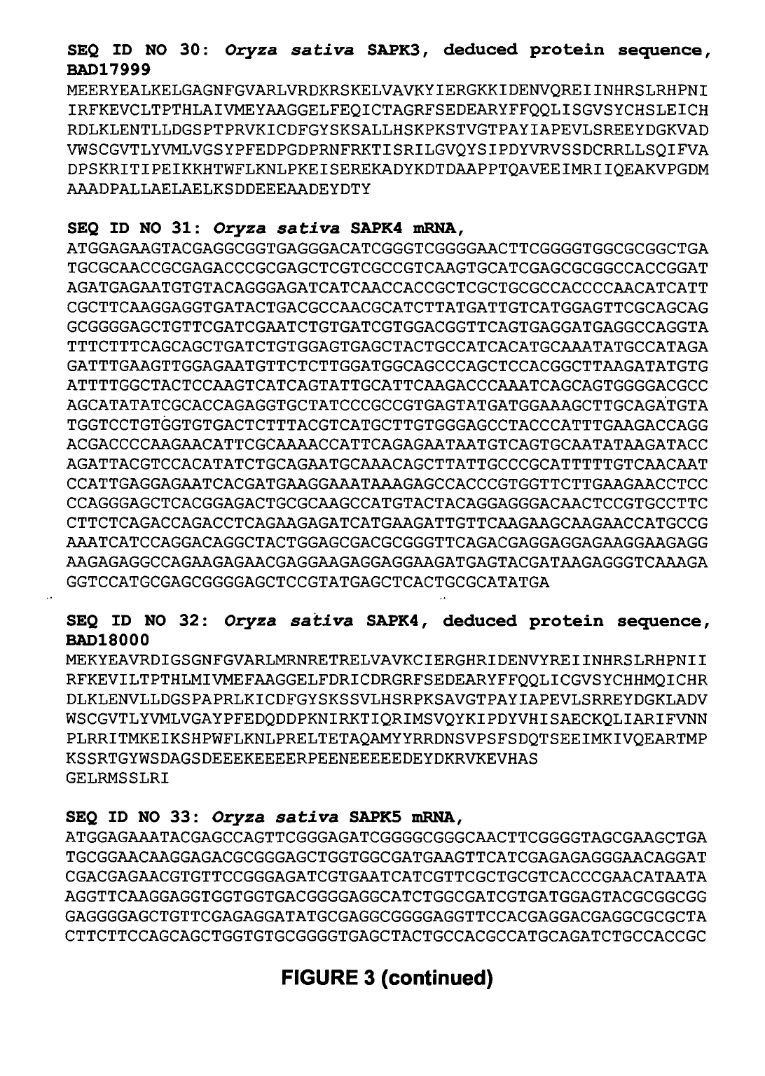

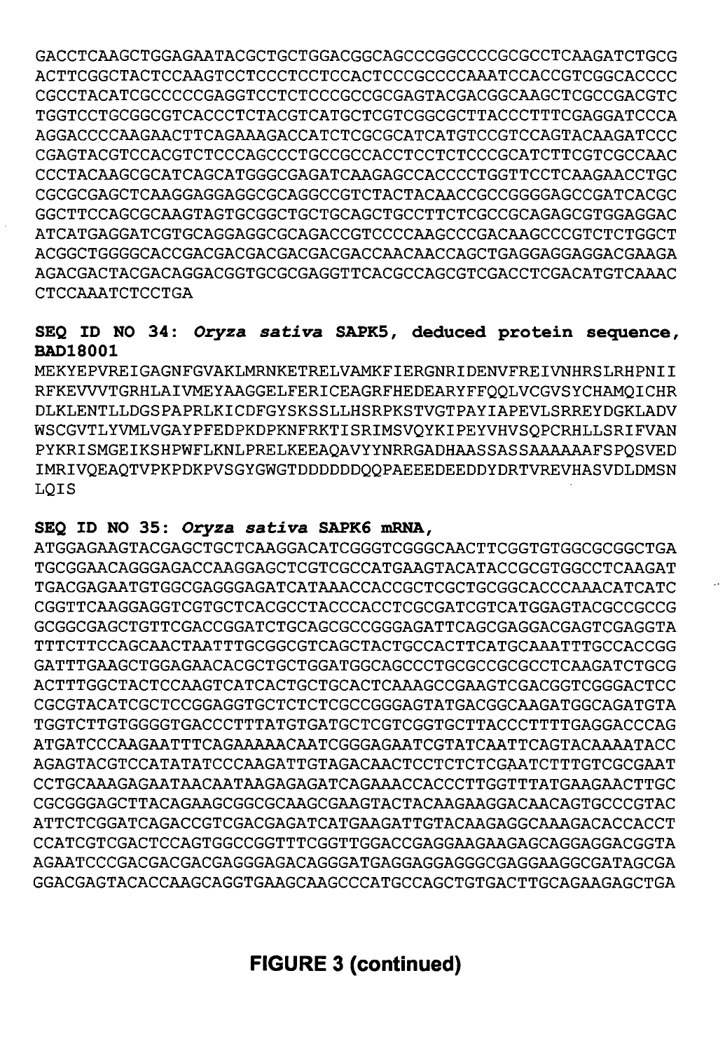

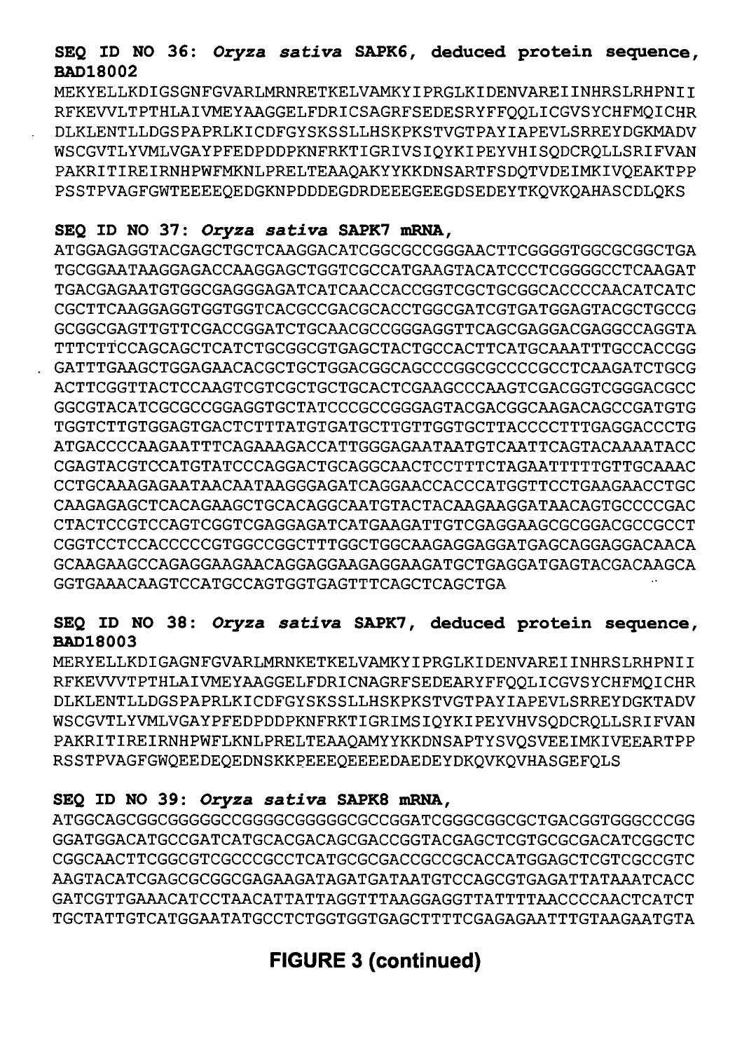

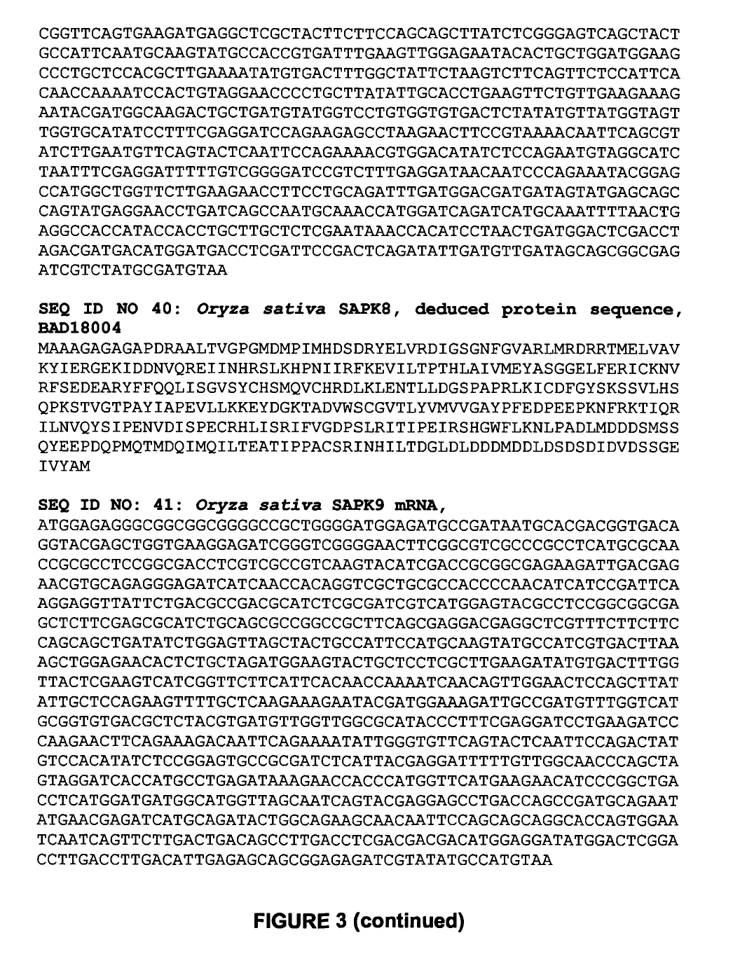

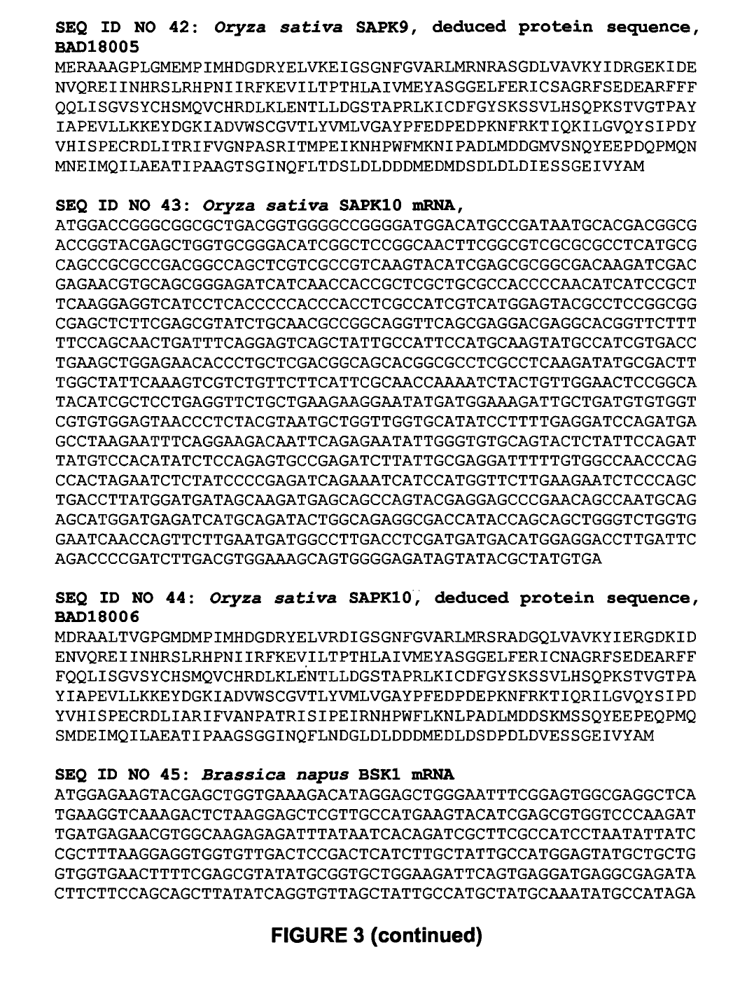

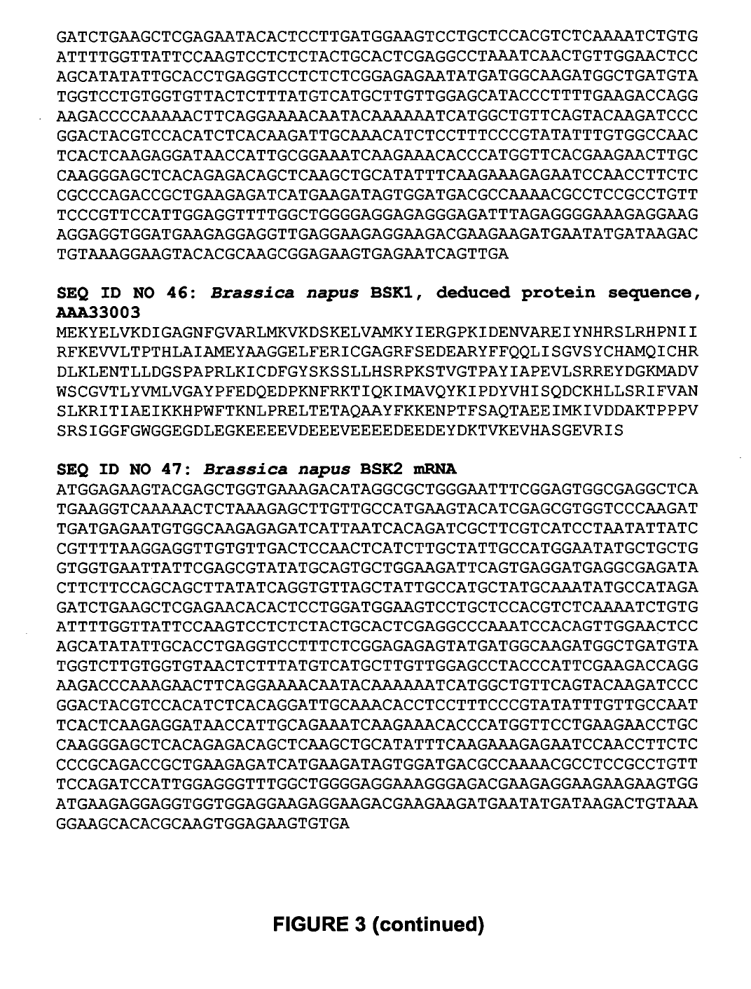

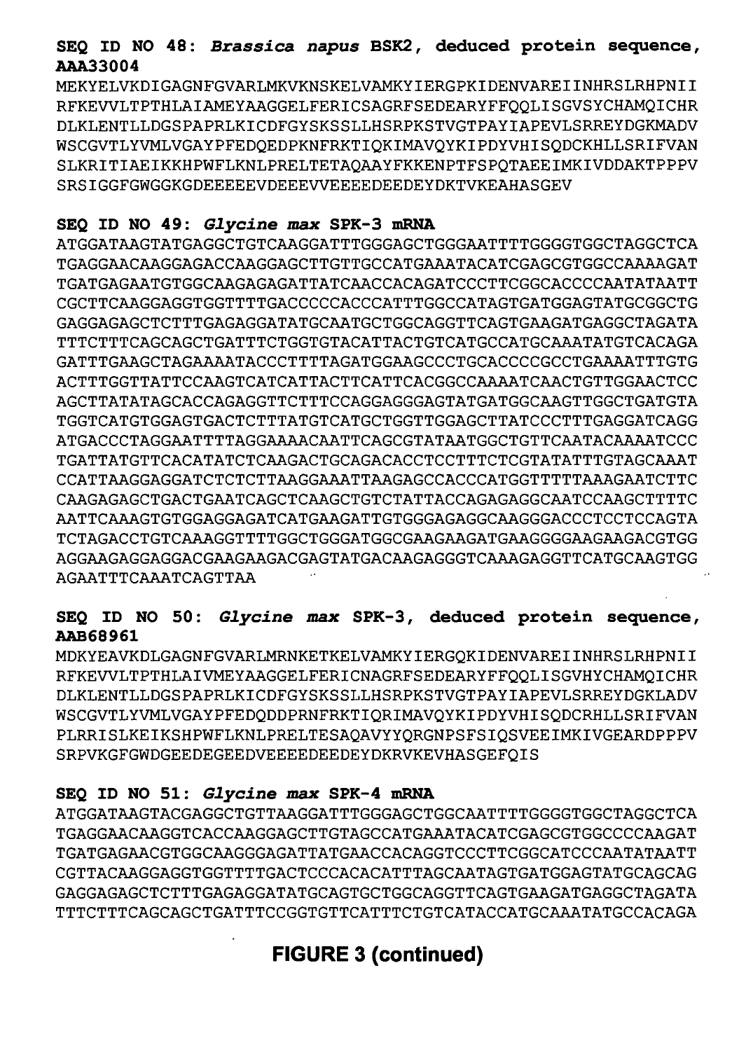

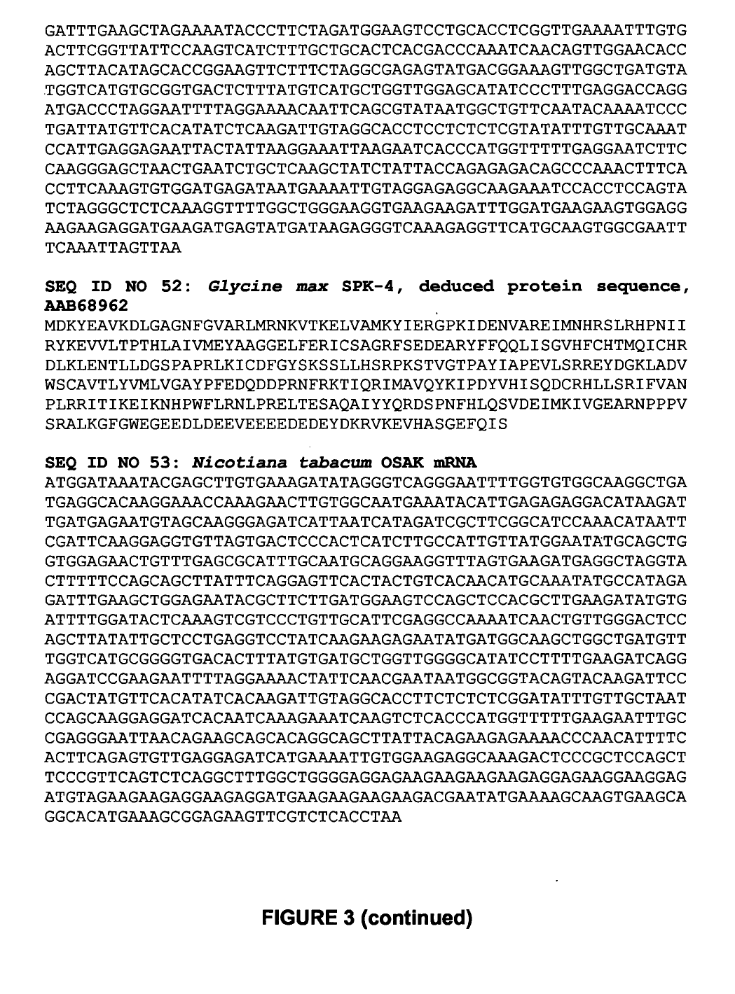

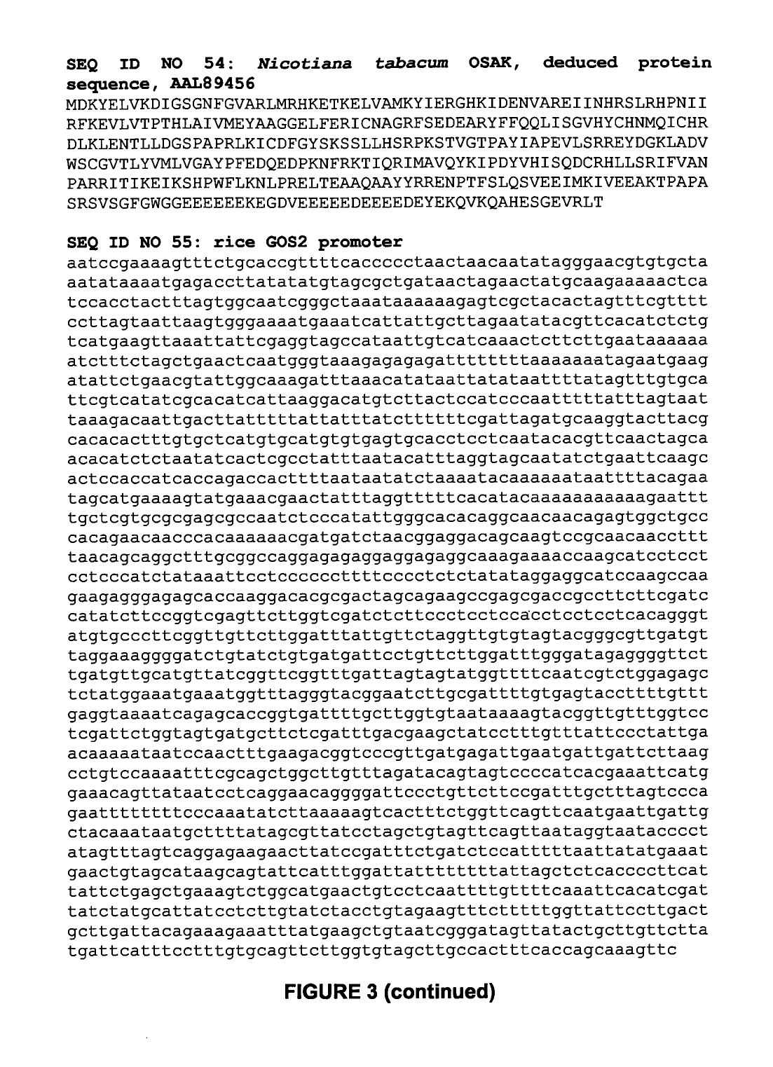

### EXAMPLES

The present invention will now be described with reference to the following examples, which are by way of illustration alone.

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols (http://www.4ulr.com/products/currentprotocols/index.html). Standard materials and methods for plant molecular work are described in Plant Molecular Biology Labfax (1993) by R. D. D. Croy, published by BIOS Scientific Publications Ltd (UK) and Blackwell Scientific Publications (UK).

### Example 1

### Gene Cloning

The Arabidopsis SnRK2 (internal code CDS0758) was amplified by PCR using as template an Arabidopsis thaliana seedling cDNA library (Invitrogen, Paisley, UK). After reverse transcription of RNA extracted from seedlings, the cDNAs were cloned into pCMV Sport 6.0. Average insert size of the bank was 1.5 kb, and the original number of clones was 1.59×10⁷ cfu. Original titer was determined to be 9.6×10⁵ cfu/ml, and after a first amplification of 6×10¹¹ cfu/ml. After plasmid extraction, 200 ng of template was used in a 50 μl PCR mix. Primers Prm02295 (SEQ ID NO: 4, sense) and Prm02296 (SEQ ID NO: 5, reverse complementary), which include the attB sites for Gateway recombination, were used for PCR amplification. PCR was performed using Hifi Taq DNA polymerase in standard conditions. A PCR fragment of 1130 bp (without the attB sites) was amplified and purified also using standard methods. The first step of the Gateway procedure, the BP reaction, was then performed, during which the PCR fragment recombines in vivo with the pDONR201 plasmid to produce, according to the Gateway® terminology, an “entry clone”, p028. Plasmid pDONR201 was purchased from Invitrogen, as part of the Gateways technology.

### Example 2

### Vector Construction and Rice Transformation

The entry done p028 was subsequently used in an LR reaction with p03069, a destination vector used for Oryza sativa transformation. This vector contained as functional elements within the T-DNA borders: a plant selectable marker; a visual marker expression cassette; and a Gateway cassette intended for LR in vivo recombination with the sequence of interest already cloned in the entry done. A rice GOS2 promoter for constitutive expression was located upstream of this Gateway cassette.

After the LR recombination step, the resulting expression vector p033 (FIG. 2) was transformed into the Agrobacterium strain LBA4404 and subsequently to Oryza sativa plants. Transformed rice plants were allowed to grow and were then examined for the parameters described in Example 3.

### Example 3

### Evaluation of Transformants: Growth Measurements

Approximately 15 to 20 independent T0 transformants were generated. The primary transformants were transferred from tissue culture chambers to a greenhouse for growing and harvest of T1 seed. Five events of which the T1 progeny segregated 3:1 for presence/absence of the transgene were retained. For each of these events, 10 T1 seedlings containing the transgene (hetero- and homo-zygotes), and 10 T1 seedlings lacking the transgene (nullizygotes), were selected by visual marker screening. The selected T1 plants were transferred to a greenhouse. Each plant received a unique barcode label to link unambiguously the phenotyping data to the corresponding plant. The selected T1 plants were grown on soil in 10 cm diameter pots under the following environmental settings: photoperiod=11.5 h, daylight intensity=30,000 lux or more, daytime temperature=28° C. or higher, night time temperature=22° C., relative humidity=60-70%. Transgenic plants and the corresponding nullizygotes were grown side-by-side at random positions. From the stage of sowing until the stage of maturity the plants were passed several times through a digital imaging cabinet. At each time point digital images (2048×1536 pixels, 16 million colours) were taken of each plant from at least 6 different angles.

The mature primary panicles were harvested, bagged, barcode-labelled and then dried for three days in the oven at 37° C. The panicles were then threshed and all the seeds collected. The filled husks were separated from the empty ones using an air-blowing device. After separation, both seed lots were then counted using a commercially available counting machine. The empty husks were discarded. The filled husks were weighed on an analytical balance and the cross-sectional area of the seeds was measured using digital imaging. This procedure resulted in the set of seed-elated parameters described below.

These parameters were derived in an automated way from the digital images using image analysis software and were analysed statistically. A two factor ANOVA (analyses of variance) corrected for the unbalanced design was used as statistical model for the overall evaluation of plant phenotypic characteristics. An F-test was carried out on all the parameters measured of all the plants of all the events transformed with that gene. The F-test was carried out to check for an effect of the gene over all the transformation events and to verify for an overall effect of the gene, also named herein “global gene effect”. If the value of the F test shows that the data are significant, than it is concluded that there is a “gene” effect, meaning that not only presence or the position of the gene is causing the effect. The threshold for significance for a true global gene effect is set at 5% probability level for the F test.

To check for an effect of the genes within an event, i.e., for a line-specific effect, a t-test was performed within each event using data sets from the transgenic plants and the corresponding null plants. “Null plants” or “null segregants” or “nullizygotes” are the plants treated in the same way as the transgenic plant, but from which the transgene has segregated. Null plants may also be described as the homozygous negative transformed plants. The threshold for significance for the t-test is set at 10% probability level. The results for some events can be above or below this threshold. This is based on the hypothesis that a gene might only have an effect in certain positions in the genome, and that the occurrence of this position-dependent effect is not uncommon. This kind of gene effect is also named herein a “line effect of the gene”. The p-value is obtained by comparing the t-value to the t-distribution or alternatively, by comparing the F-value to the F-distribution. The p-value then gives the probability that the null hypothesis (i.e., that there is no effect of the transgene) is correct.

The data obtained in the first experiment were confirmed in a second experiment with T2 plants. Three lines that had the correct expression pattern were selected for further analysis. Seed batches from the positive plants (both hetero- and homozygotes) in T1, were screened by monitoring marker expression. For each chosen event, the heterozygote seed batches were then retained for T2 evaluation. Within each seed batch an equal number of positive and negative plants were grown in the greenhouse for evaluation.

A total number of 120 SnRK2 transformed plants were evaluated in the T2 generation, that is 40 plants per event of which 20 positives for the transgene, and 20 negatives.

Because two experiments with overlapping events have been carried out, a combined analysis was performed. This is useful to check consistency of the effects over the two experiments, and if this is the case, to accumulate evidence from both experiments in order to increase confidence in the conclusion. The method used was a mixed-model approach that takes into account the multilevel structure of the data (i.e. experiment-event-segregants). P-values are obtained by comparing likelihood ratio test to chi square distributions.

### Example 4

### Evaluation of Transformants: Measurement of Yield-Related Parameters

Upon analysis of the seeds as described above, the inventors found that plants transformed with the SnRK2 gene construct had a higher biomass (expressed as Total Areamax) and an increased Thousand Kernel Weight (TKW) compared to plants lacking the SnRK2 transgene. Positive results obtained for plants in the T1 generation (increased Thousand Kernel Weight and a biomass increase of 9% (p-value 0.0309)) were again obtained in the T2 generation. In Table 4, data show the overall % increases for biomass and TKW, calculated from the data of the individual lines of the T2 generation, and the respective p-values from the F-test These T2 data were re-evaluated in a combined analysis with the results for the T1 generation, and the obtained p-values show that the observed effects were significant.

TABLE 4

| | |
|---|---|
| T2 generation | Combined analysis |

| | % difference | p-value | p-value |
|---|---|---|---|
| Total Areamax | +7 | 0.0158 | 0.0006 |
| TKW | +2 | 0.0107 | 0.0292 |

### Aboveground Biomass:

Plant aboveground area was determined by counting the total number of pixels from aboveground plant parts discriminated from the background. This value was averaged for the pictures taken on the same time point from the different angles and was converted to a physical surface value expressed in square mm by calibration (Total Areamax). Experiments show that the aboveground plant area measured this way correlates with the biomass of plant parts above ground. There was a significant increase in above ground biomass in the T1 generation, and this was confirmed in the T2 generation (with p-values of respectively 0.0309 in T1 and 0.0158 in T2). Also the combined analysis showed that the obtained increase in biomass was highly significant (p-value of 0.0006).

### Thousand Kernel Weight:

The Thousand Kernel Weight (TKW) is extrapolated from the number of filled seeds counted and their total weight. There was a tendency for increased TKW in the T1 generation, and in the T2 generation, it was shown that the increase was a true overall effect and was significant. In particular, 2 of the four tested T2 lines showed a significantly increased TKW.

1. A method for improving growth characteristics of a plant, relative to a corresponding wild type plant, comprising increasing activity of an SnRK2 polypeptide or a homologue thereof and/or by increasing expression of an SnRK2 encoding nucleic acid, and optionally selecting for plants having improved growth characteristics.

2. The method of claim 1, wherein said increased activity and/or increased expression is effected by introducing a genetic modification in the locus of a gene encoding an SnRK2 polypeptide or a homologue thereof.

3. The method of claim 2, wherein said genetic modification is effected by one of site-directed mutagenesis, homologous recombination, TILLING, directed evolution and T-DNA activation.

4. A method for improving plant growth characteristics, relative to corresponding wild type plants, comprising introducing and expressing in a plant an SnRK2 nucleic acid molecule or a functional variant thereof.

5. The method of claim 4, wherein said functional variant is a portion of an SnRK2 nucleic acid molecule or a sequence capable of hybridising to an SnRK2 nucleic acid molecule and wherein said functional variant comprises a kinase domain, the conserved sequence signature of SEQ ID NO: 6 and an acidic C-terminal domain.

6. The method of claim 4, wherein said SnRK2 nucleic acid molecule or functional variant thereof is overexpressed in a plant.

7. The method of claim 4, wherein said SnRK2 nucleic acid molecule or functional variant thereof is of plant origin.

8. The method of claim 4, wherein said functional variant encodes an orthologue or paralogue of SnRK2.

9. The method of claim 4, wherein said SnRK2 nucleic acid molecule or functional variant thereof is operably linked to a constitutive promoter.

10. The method according to claim 9, wherein said constitutive promoter is a GOS2 promoter.

11. The method of claim 1, wherein said improved plant growth characteristic is increased yield.

12. The method according to claim 11, wherein said increased yield is increased biomass and/or increased seed yield.

13. The method according to claim 12, wherein said increased seed yield is selected from any one or more of (i) increased seed biomass; (ii) increased number of (filled) seeds; (iii) increased seed size; (iv) increased seed volume; (v) increased harvest index (HI); and (vi) increased thousand kernel weight (TKW).

14. A plant or plant cell obtainable by the method of claim 1.

15. A construct comprising:
(i) an SnRK2 nucleic acid molecule or functional variant thereof;
(ii) one or more control sequence capable of driving expression of the nucleic acid sequence of (i); and optionally
(iii) a transcription termination sequence.

16. The construct according to claim 15, wherein said control sequence is a constitutive promoter.

17. The construct according to claim 16, wherein said constitutive promoter is a GOS2 promoter.

18. A plant or plant cell transformed with the construct according to claim 15.

19. A method for the production of a transgenic plant having improved growth characteristics, which method comprises:
(i) introducing into a plant an SnRK2 nucleic acid molecule or functional variant thereof; and
(ii) cultivating the plant cell under conditions promoting plant growth and development.

20. A transgenic plant or plant cell having improved growth characteristics relative to a corresponding wild type plant, resulting from an SnRK2 nucleic acid molecule or functional variant thereof introduced into said plant or plant cell, or resulting from a genetic modification in the locus of a gene encoding an SnRK2 polypeptide or a homologue thereof.

21. The transgenic plant or plant cell according to claim 20, wherein said plant is a monocotyledonous plant, and wherein said plant cell is derived from a monocotyledonous plant.

22. A harvestable part, and/or product directly derived therefrom, of a plant according to claim 20.

23. A harvestable part according to claim 22, wherein said harvestable part is a seed.

24. (canceled)

25. The method of claim 12, wherein said increased seed yield comprises at least increased thousand kernel weight.

26. A method of selecting a plant with improved growth characteristics comprising utilizing an SnRK2 nucleic acid molecule or functional variant thereof as a molecular marker.

27. A composition comprising an SnRK2 nucleic acid molecule or functional variant thereof for improving growth characteristics of plants, for use as a growth regulator.

28. A composition comprising an SnRK2 protein or a homologue thereof for improving growth characteristics of plants, for use as a growth regulator.

29. The method of claim 4, wherein said SnRK2 nucleic acid molecule or functional variant thereof is from a dicotyledonous plant.

30. The method of claim 29, wherein said dicotyledonous plant is from the family Brassicaceae.

31. The method of claim 29, wherein said dicotyledonous plant is Arabidopsis thaliana.

32. The transgenic plant or plant cell of claim 21, wherein said monocotyledonous plant is selected from the group consisting of sugar cane, rice, maize, wheat, barley, millet, rye oats, and sorghum.


SEQUENCE LISTING (55 sequences)

SEQ ID NO: 1 - DNA, 1092 bp, Arabidopsis thaliana
atggacaagtacgagctggtgaaagacataggtgctgggaattttggagttgccaggctcatgaaggtcaaaaactctaaggaacttgttgccatgaagtacatcgagcgtggtcctaagattgatgagaatgtggcaagagagatcattaatcacagatcacttcgccatccgaatataatccggttcaaggaggtggtgttgactccaacccatcttgccattgccatggaatatgctgctggtggtgaactattcgagcgtatatgcagtgctggaagatttagtgaggatgaggcgagatatttcttccagcagcttatatcaggtgttagctattgccatgctatgcaaatatgccatagagatctgaagctcgagaatacgctcttggatggaagtcctgctccacgtctgaaaatctgtgattttggttattccaagtcctctctgctgcactctaggcccaaatcaacagttggaactccagcatatattgcacctgaggtcctttctcgaagagaatatgatggcaagatggctgatgtatggtcttgtggtgtgactctttatgtcatgctggttggagcatacccatttgaagaccaggaagaccccaagaacttcaggaaaacaatacaaaaaataatggctgtccagtacaagatcccggactacgtccatatctcacaggattgtaaaaatctcctttcccgtatatttgtcgccaattcactcaagaggatcaccattgcagaaatcaagaaacattcatggttcctaaagaatttgccaagggaactcacagagacagctcaagctgcatatttcaagaaagagaacccaaccttctcccttcagaccgttgaagagatcatgaagatagtggctgacgccaaaacaccgcctcctgtttcccgatccatcggaggttttggctggggaggaaatggggatgcagatggaaaagaggaagatgcagaagacgtggaggaggaagaggaggaggtggaagaagaggaagacgatgaggatgaatacgataagactgtaaaggaagtacacgcaagtggagaagtgagaataagttga

SEQ ID NO: 2 - protein, 363 aa, Arabidopsis thaliana
MDKYELVKDIGAGNFGVARLMKVKNSKELVAMKYIERGPKIDENVAREIINHRSLRHPNIIRFKEVVLTPTHLAIAMEYAAGGELFERICSAGRFSEDEARYFFQQLISGVSYCHAMQICHRDLKLENTLLDGSPAPRLKICDFGYSKSSLLHSRPKSTVGTPAYIAPEVLSRREYDGKMADVWSCGVTLYVMLVGAYPFEDQEDPKNFRKTIQKIMAVQYKIPDYVHISQDCKNLLSRIFVANSLKRITIAEIKKHSWFLKNLPRELTETAQAAYFKKENPTFSLQTVEEIMKIVADAKTPPPVSRSIGGFGWGGNGDADGKEEDAEDVEEEEEEVEEEEDDEDEYDKTVKEVHASGEVRIS

SEQ ID NO: 3 - DNA, 3121 bp, Arabidopsis thaliana
caaacatgtatggatcgcatggaaacttgtgggtccctctttcttttaaaaaatctcgtattaattaaataaatggaaaaaaaaacatgatgggagtttgtttaggcaggggagattcttcttcattctcatcattatttctctattaatttcaccccaaaaaagaaaaaagaaaaattccaacaagaaaaaaaaaagaaaaagaaagttgattcttcgcttaggcttgaaatctctccaatccaaatctcaaattaaccttccatcgtcatctctttccctttttttttcccactttctttgcgaatcgcgagatctcggaatcgcatccttgattttgggatactgttttttttttttttaatcttgtttcattttcacgtgaaattcttagctgctagaactggacttgaatttcaacgagaattttggagattttttttttgtttgggtttttcctttctgttttgtgtgtttggaattagggttgtcgagcgagaatggacaagtacgagctggtgaaagacataggtgctgggaattttggagttgccaggctcatgaaggtcaaaaactctaaggaacttgttgccatgaagtacatcgagcgtggtcctaaggtatattcctctctgtttttgtgttttcattgctctccatgagctggtgatcctatacccagatatgcataattggaatgaattgctattaagcagaagagtcgatttttttttgtaaatttcttatcgttagctgattgggtgtttaacgtaacgttagttatcttgtagttgtaatatttttcctgaaaagatttgtacaatgagtatttgctttgtttgttttttttgatacagattgatgagaatgtggcaagagagatcattaatcacagatcacttcgccatccgaatataatccggttcaaggaggttagtgaatttcttgttgcttgacatgggtggtgttcttgctatgaaaaagtttgttgataatctcttatatctttcatcttgcattccttgttgggtttatggattttataggtggtgttgactccaacccatcttgccattgccatggaatatgctgctggtggtgaactattcgagcgtatatgcagtgctggaagatttagtgaggatgaggtgagcttgccatttgaaaaattgtgctgtgcttttgcgaatatgaaattactagtatttaggataatctgcatggtctttggaaagattaggaggaagggaacaagagaaaacatgtgaacctccttttatttagtatcaggcattaaacagttagggtctacgttctaatcctttctctcttttccaggcgagatatttcttccagcagcttatatcaggtgttagctattgccatgctatggtaatgtagagacaatgacttaagcaaaatttacttatccattggctgtttgaagtcgtttttttttaatcatgtgttgactattttgttgcagcaaatatgccatagagatctgaagctcgagaatacgctcttggatggaagtcctgctccacgtctgaaaatctgtgattttggttattccaaggtctgacactaaaaaaaaatccaagttccccccttgtcgacgagatcctcttttgtgatttgttattctcttttttttagtcctctctgctgcactctaggcccaaatcaacagttggaactccagcatatattgcacctgaggtcctttctcgaagagaatatgatggcaaggtaatcaagcatcatgcacaatgcaatgaacttccataaacccatgagtatttatgatattgtcatgctctttacatttttacttttgaatttaaaaagtcatctttgtggaagtcgctaagatttgaagcattttttcttctttcagatggctgatgtatggtcttgtggtgtgactctttatgtcatgctggttggagcatacccatttgaagaccaggaagaccccaagaacttcaggaaaacaatacaagtaggtttcttttttgaagccatgtatctgcatatctcgctttcgccacatcctattcgtcaatgtgtgatcttgttatacagaaaataatggctgtccagtacaagatcccggactacgtccatatctcacaggattgtaaaaatctcctttcccgtatatttgtcgccaattcactcaaggtatacatcaatcaactgaactaaatgttttcaaagatgccttttgatttttctgaacaattgagctacttgttgtttcgtagaggatcaccattgcagaaatcaagaaacattcatggttcctaaagaatttgccaagggaactcacagagacagctcaagctgcatatttcaagaaagagaacccaaccttctcccttcagaccgttgaagagatcatgaagatagtggctgacgccaaaacaccgcctcctgtttcccgatccatcggaggttttggctggggaggaaatggggatgcagatggaaaagaggaagatgcagaagacgtggaggaggaagaggaggaggtggaagaagaggaagacgatgaggatgaatacgataagactgtaaaggaagtacacgcaagtggagaagtgagaataagttgatattttggtttttggtctgtgtaagaaagaagtcgtcgttggtttgttgaaactgaaaagtctctgttctcgtgtttgcctttacaatgctttggctaaggttttggttctggttttggagatttgtaaaatttgcagtataagatgaacaaacagagaggttgatgatgagaatgagtcctttgctacgcatggtactatgaacattgtgacctccaataaatatttttgtaaattagattttattttccgaaaagattcatgtatttgatttttggatttcttatttttattttttttcgttccttatcatttttttgaaaatgcaaatctataaaatacaaatgtcaacaaaaaatcaaattgaaatgttcggaattcaaaaataattgttttcttttgtttttttgtttctgatgcgaaatgtgaatatattagagggaaaatatcccgccattaggaaaccggataatcttctacggccttgagctcaagtcggt

SEQ ID NO: 4 - DNA, 54 bp, Artificial sequence
  forward primer prm02295
ggggacaagtttgtacaaaaaagcaggcttcacaatggacaagtacgagctggt

SEQ ID NO: 5 - DNA, 51 bp, Artificial sequence
  reverse primer prm02296
ggggaccactttgtacaagaaagctgggtcgacgacttctttcttacacag

SEQ ID NO: 6 - protein, 9 aa, Artificial sequence
  conserved signature sequence
WXXKXXXXX

SEQ ID NO: 7 - DNA, 1086 bp, Arabidopsis thaliana
atggacaagtacgagcttgttaaagacatcggtgctgggaattttggagtggcgaggctcatgagagtcaaaaactccaaggaactcgttgctatgaagtacatcgagcgtggacctaagattgatgagaacgtggcgagagagattattaaccacagatcacttcgtcatcccaatattatccggtttaaggaggtggttttgacaccaacgcacatcgccattgctatggaatatgctgctggcggtgagctatttgagcgtatatgtagcgctggaagattcagtgaggatgaggcaagatactttttccagcagcttatctcaggagtcagctattgtcatgctatgcaaatatgccacagagatctgaagcttgaaaataccctcttagatggaagtcctgctccacgcctgaagatctgtgattttggttattccaagtcctcactgttgcactctatgcccaaatcaactgttggaactccagcatatattgcacctgaggttctttctcgcggagagtatgatggcaagatggctgatgtatggtcttgtggtgtgactctttatgtcatgctggtgggagcatacccatttgaagaccaagaggatcccaaaaacttcaaaaaaacaatacaaagaataatggctgtcaagtacaagatcccggactatgtccatatctcacaagattgcaaacatctcctctcccgtatatttgtcaccaactcgaataagaggattacgataggtgacatcaagaaacatccatggttcctaaagaacctgccaagggaacttacagaaatagctcaagctgcatacttcaggaaagagaacccgacattctcactccaaagcgtcgaagagataatgaagattgtggaagaggcaaaaactccagctcgtgtttctcggtcgattggagcatttgggtggggaggaggagaagatgccgagggcaaggaggaagatgcagaggaagaagttgaggaagtagaagaagaagaagacgaagaagatgagtatgataagacggtgaagcaagtgcatgctagcatgggagaagtccgagtcagttaa

SEQ ID NO: 8 - protein, 361 aa, Arabidopsis thaliana
MDKYELVKDIGAGNFGVARLMRVKNSKELVAMKYIERGPKIDENVAREIINHRSLRHPNIIRFKEVVLTPTHIAIAMEYAAGGELFERICSAGRFSEDEARYFFQQLISGVSYCHAMQICHRDLKLENTLLDGSPAPRLKICDFGYSKSSLLHSMPKSTVGTPAYIAPEVLSRGEYDGKMADVWSCGVTLYVMLVGAYPFEDQEDPKNFKKTIQRIMAVKYKIPDYVHISQDCKHLLSRIFVTNSNKRITIGDIKKHPWFLKNLPRELTEIAQAAYFRKENPTFSLQSVEEIMKIVEEAKTPARVSRSIGAFGWGGGEDAEGKEEDAEEEVEEVEEEEDEEDEYDKTVKQVHASMGEVRVS

SEQ ID NO: 9 - DNA, 1083 bp, Arabidopsis thaliana
atggacaagtatgaggttgtgaaggatttgggagctggaaattttggtgtggctcgtcttcttagacacaaagagaccaaagagctcgttgctatgaaatacattgagagaggtcgcaagattgatgagaatgtggcaagagagattatcaatcatagatcacttaggcatcctaatatcatcagattcaaggaggtgattctgactccaactcatcttgcaattgtaatggagtatgcttctggaggagagctctttgaaagaatctgtaatgctggtagattcagtgaagctgaggctagatacttctttcagcagctgatttgtggcgtggattactgtcattcactgcaaatatgtcatagagatttgaagcttgagaatacactgcttgatggtagtccagccccgcttttgaaaatctgtgattttggttactccaagtcatctctgcttcactctagacctaaatcaactgttggtactccagcttatatcgcacctgaagttctttcccgaagagaatatgacggaaagcatgcggatgtttggtcctgtggtgtgactctttatgtgatgttagttggaggttatccgtttgaagacccggatgatccgagaaacttcaggaaaacaatccaacgtataatggctgtccagtacaagatcccggattacgttcatatatcgcaggagtgcagacaccttctctctcgcatatttgtcactaattcagctaagagaatcacacttaaagagatcaagaagcatccatggtacttaaagaacttgccaaaggagcttacagagcctgctcaagcggcgtactacaagagagaaaccccaagcttttccctccaaagcgtagaggacataatgaagatcgttggagaagccaggaatccagctccgtcttctaatgccgtcaagggctttgatgatgatgaggaagatgtggaggacgaggttgaagaagaagaagaagaagaagaagaagaggaggaagaagaggaagaggaagaagatgaatacgagaagcatgttaaagaggcccattcttgtcaagagcctcccaaagcttaa

SEQ ID NO: 10 - protein, 360 aa, Arabidopsis thaliana
MDKYEVVKDLGAGNFGVARLLRHKETKELVAMKYIERGRKIDENVAREIINHRSLRHPNIIRFKEVILTPTHLAIVMEYASGGELFERICNAGRFSEAEARYFFQQLICGVDYCHSLQICHRDLKLENTLLDGSPAPLLKICDFGYSKSSLLHSRPKSTVGTPAYIAPEVLSRREYDGKHADVWSCGVTLYVMLVGGYPFEDPDDPRNFRKTIQRIMAVQYKIPDYVHISQECRHLLSRIFVTNSAKRITLKEIKKHPWYLKNLPKELTEPAQAAYYKRETPSFSLQSVEDIMKIVGEARNPAPSSNAVKGFDDDEEDVEDEVEEEEEEEEEEEEEEEEEEDEYEKHVKEAHSCQEPPKA

SEQ ID NO: 11 - DNA, 1062 bp, Arabidopsis thaliana
atggacaagtatgacgttgtcaaggatctgggagctggaaatttcggtgtggctcgccttctcaggcacaaggacaccaaagagcttgttgccatgaaatacatcgagagaggtcgcaagatagatgagaacgtggcgagagagattattaatcacagatcacttaaacatcctaatatcatccggttcaaggaggtgatcctgacacctactcatcttgctattgtgatggagtatgcttctggaggagagctctttgatcgaatctgtactgccggtagatttagtgaagctgaggctaggtacttctttcaacagctgatttgtggtgttgattactgccattccttgcaaatatgtcatagagacctgaagcttgagaacacactgctcgatgggagccctgctccgcttttgaaaatctgtgattttggttactctaagtcatctatactacattctaggcctaaatcaactgttggaactccagcttacatagcacctgaagttctttcacggagagaatatgatggcaagcacgcggatgtgtggtcatgtggagtaaccctttatgtgatgctggtgggagcttacccgtttgaggaccctaatgatccaaaaaacttcaggaaaacaatccagcgcataatggctgtacaatacaagatcccggactatgttcacatatctcaggaatgcaaacatcttctctctcgcatattcgtcactaactctgctaagagaatcacacttaaggagatcaagaatcatccgtggtacttgaagaatttgccaaaggagctgctagagtcggctcaagcggcgtattacaagagagacacaagcttctctcttcaaagcgtagaggacataatgaagatagttggagaagcaaggaatccagctccatcaactagtgctgtcaaaagctcgggctcaggagctgatgaagaagaggaagaggacgttgaagctgaagtggaagaggaagaagatgatgaagacgaatacgagaagcatgtcaaagaggcacagtcttgtcaagagtctgacaaagcttaa

SEQ ID NO: 12 - protein, 353 aa, Arabidopsis thaliana
MDKYDVVKDLGAGNFGVARLLRHKDTKELVAMKYIERGRKIDENVAREIINHRSLKHPNIIRFKEVILTPTHLAIVMEYASGGELFDRICTAGRFSEAEARYFFQQLICGVDYCHSLQICHRDLKLENTLLDGSPAPLLKICDFGYSKSSILHSRPKSTVGTPAYIAPEVLSRREYDGKHADVWSCGVTLYVMLVGAYPFEDPNDPKNFRKTIQRIMAVQYKIPDYVHISQECKHLLSRIFVTNSAKRITLKEIKNHPWYLKNLPKELLESAQAAYYKRDTSFSLQSVEDIMKIVGEARNPAPSTSAVKSSGSGADEEEEEDVEAEVEEEEDDEDEYEKHVKEAQSCQESDKA

SEQ ID NO: 13 - DNA, 1089 bp, Arabidopsis thaliana
atggatcgaccagcagtgagtggtccaatggatttgccgattatgcacgatagtgataggtatgaactcgtcaaggatattggctccggtaattttggagttgcgagattgatgagagacaagcaaagtaatgagcttgttgctgttaaatatatcgagagaggtgagaagatagatgaaaatgtaaaaagggagataatcaaccacaggtccttaagacatcccaatatcgttagattcaaagaggttatattaacaccaacccatttagccattgttatggaatatgcatctggaggagaacttttcgagcgaatctgcaatgcaggccgcttcagcgaagacgaggcgaggtttttcttccagcaactcatttcaggagttagttactgtcatgctatgcaagtatgtcaccgagacttaaagctcgagaatacgttattagatggtagcccggcccctcgtctaaagatatgtgatttcggatattctaagtcatcagtgttacattcgcaaccaaaatcaactgttggaactcctgcttacatcgctcctgaggttttactaaagaaagaatatgatggaaaggttgcagatgtttggtcttgtggggttactctgtatgtcatgctggttggagcatatcctttcgaagatcccgaggaaccaaagaatttcaggaaaactatacatagaatcctgaatgttcagtatgctattccggattatgttcacatatctcctgaatgtcgccatttgatctccagaatatttgttgctgaccctgcaaagaggatatcaattcctgaaataaggaaccatgaatggtttctaaagaatctaccggcagatctaatgaacgataacacgatgaccactcagtttgatgaatcggatcaaccgggccaaagcatagaagaaattatgcagatcattgcagaagcaactgttcctcctgcaggcactcagaatctgaaccattacctcacaggaagcttggacatagatgacgatatggaggaagacttagagagcgaccttgatgatcttgacatcgacagtagcggagagattgtgtacgcaatgtga

SEQ ID NO: 14 - protein, 362 aa, Arabidopsis thaliana
MDRPAVSGPMDLPIMHDSDRYELVKDIGSGNFGVARLMRDKQSNELVAVKYIERGEKIDENVKREIINHRSLRHPNIVRFKEVILTPTHLAIVMEYASGGELFERICNAGRFSEDEARFFFQQLISGVSYCHAMQVCHRDLKLENTLLDGSPAPRLKICDFGYSKSSVLHSQPKSTVGTPAYIAPEVLLKKEYDGKVADVWSCGVTLYVMLVGAYPFEDPEEPKNFRKTIHRILNVQYAIPDYVHISPECRHLISRIFVADPAKRISIPEIRNHEWFLKNLPADLMNDNTMTTQFDESDQPGQSIEEIMQIIAEATVPPAGTQNLNHYLTGSLDIDDDMEEDLESDLDDLDIDSSGEIVYAM

SEQ ID NO: 15 - DNA, 1020 bp, Arabidopsis thaliana
atggagaagtatgagatggtgaaggatttaggatttggtaatttcggattggctcggcttatgcgtaataagcaaacaaacgagcttgtggctgtcaaattcatcgatcgaggctacaagatagatgagaacgttgcaagagaaataatcaatcatagagctctcaaccatccgaatattgttcggtttaaagaggttgttttaactccgacacatcttggaatagtaatggagtatgcagctggaggagaactgttcgagcggatatctagcgtgggtcgatttagcgaagctgaggcaagatatttctttcaacaactcatttgtggagtccattacttacatgcattgcaaatatgccatagagatctgaaattagaaaacacattgcttgatggaagcccagcaccacgtttaaaaatttgtgattttggctactcaaagtcttctgttctgcactccaacccaaaatcaacggtgggaactccggcatatatagcaccggaagttttttgtcgatcggaatacgacggaaagtcagttgatgtgtggtcttgtggagtggccctctatgttatgttggtaggagcttatccattcgaagaccctaaagaccctcgcaatttccgaaaaactgttcagaaaataatggccgtaaactacaagattccaggatatgttcacatatccgaagactgcagaaagttactatctcgtatatttgttgccaatccgttacatagaagtacgcttaaagagattaagagtcatgcatggttcctaaagaatttgccaagagaattaaaggagccagcacaagcaatctattaccaaaggaatgttaatcttattaatttttctcctcaaagagtagaggagattatgaagatagttggtgaggcaagaaccattccaaacctttctcgcccggtcgaatcgcttggatcagataaaaaagatgatgatgaagaagaatatttggatgctaatgatgaagaatggtatgatgattacgcatag

SEQ ID NO: 16 - protein, 339 aa, Arabidopsis thaliana
MEKYEMVKDLGFGNFGLARLMRNKQTNELVAVKFIDRGYKIDENVAREIINHRALNHPNIVRFKEVVLTPTHLGIVMEYAAGGELFERISSVGRFSEAEARYFFQQLICGVHYLHALQICHRDLKLENTLLDGSPAPRLKICDFGYSKSSVLHSNPKSTVGTPAYIAPEVFCRSEYDGKSVDVWSCGVALYVMLVGAYPFEDPKDPRNFRKTVQKIMAVNYKIPGYVHISEDCRKLLSRIFVANPLHRSTLKEIKSHAWFLKNLPRELKEPAQAIYYQRNVNLINFSPQRVEEIMKIVGEARTIPNLSRPVESLGSDKKDDDEEEYLDANDEEWYDDYA

SEQ ID NO: 17 - DNA, 1086 bp, Arabidopsis thaliana
atggatcgagctccggtgaccacaggaccgttggatatgccgattatgcacgacagtgatcgatatgacttcgttaaggatattggttctggtaatttcggtgttgctcgtcttatgagagataaactcactaaagagcttgttgctgtcaagtacatcgagagaggagacaagattgatgaaaatgttcaaagggagatcattaaccacaggtcactaaggcatcctaatattgtcagatttaaagaggtcattttgacgccgactcatctggctatcataatggaatatgcttctggcggtgaactttacgagcggatttgcaatgcaggacggtttagtgaagatgaggctcggttcttctttcagcagcttctatctggagtcagttattgtcattcgatgcaaatttgccatcgtgacctgaagctagagaatacattgttggatggaagtcctgctcctcgattaaaaatttgtgattttggatattcaaagtcttctgttcttcattcacaaccaaagtcaactgttggtactcctgcatacatcgctccagaggtactgcttcgtcaggaatatgatggcaagattgcagatgtatggtcatgtggtgtgaccttatacgtcatgttggttggagcgtatccgttcgaagatccagaagagccaagagactatcggaaaacaatacagagaatccttagcgttaaatactcaatccctgatgacatacggatatcacctgaatgctgtcatcttatttcaagaatcttcgtggctgatcccgctaccagaataagcataccagagatcaaaacccatagttggttcttgaagaatctccctgctgatctaatgaacgagagcaacacaggaagccagttccaggagcctgaacaaccaatgcaaagccttgacacaatcatgcaaatcatctctgaagccacaattcccgctgttcgaaaccgttgcctagacgatttcatgactgacaatcttgatcttgacgatgacatggatgactttgactctgaatctgaaatcgacattgacagtagcggagagatagtttacgctctctaa

SEQ ID NO: 18 - protein, 361 aa, Arabidopsis thaliana
MDRAPVTTGPLDMPIMHDSDRYDFVKDIGSGNFGVARLMRDKLTKELVAVKYIERGDKIDENVQREIINHRSLRHPNIVRFKEVILTPTHLAIIMEYASGGELYERICNAGRFSEDEARFFFQQLLSGVSYCHSMQICHRDLKLENTLLDGSPAPRLKICDFGYSKSSVLHSQPKSTVGTPAYIAPEVLLRQEYDGKIADVWSCGVTLYVMLVGAYPFEDPEEPRDYRKTIQRILSVKYSIPDDIRISPECCHLISRIFVADPATRISIPEIKTHSWFLKNLPADLMNESNTGSQFQEPEQPMQSLDTIMQIISEATIPAVRNRCLDDFMTDNLDLDDDMDDFDSESEIDIDSSGEIVYAL

SEQ ID NO: 19 - DNA, 1032 bp, Arabidopsis thaliana
atggagaggtacgaaatagtgaaggatattgggtctggtaacttcggagtagcaaagcttgttcgtgacaaattttccaaagagcttttcgctgttaagttcatcgagcgaggccaaaagattgatgaacatgtacaaagagaaatcatgaaccataggtcgctgatccatcccaatataataagattcaaggaggttttattgacggcaacacatttggcgttagtaatggaatacgccgccggaggagaactgttcggaagaatctgcagcgccggaagattcagtgaagacgaggcaaggtttttctttcagcagcttatatcaggagttaattactgtcacagtcttcaaatatgccatagagatttaaagctagagaacacgttacttgatggaagcgaagcgccacgtgtaaagatttgcgactttggatattcaaaatcaggagttcttcattcgcaaccaaagacaacagtaggaacacctgcttacattgcacctgaagtgctctccacgaaagagtatgacggcaaaatcgctgatgtttggtcttgtggagtcactttgtatgttatgcttgttggtgcttatccttttgaagatccttctgatcctaaagattttcggaagacgatcggtcggattctcaaagctcagtatgctattcctgattatgttcgagtttcggatgaatgcagacatcttctctctcggatattcgttgccaaccctgaaaagagaataacaatagaggagataaagaatcattcttggtttctcaagaacttgccggtagagatgtatgaaggatcattgatgatgaatggtccatcgactcagacagtagaagagatagtgtggatcattgaagaagctcggaaacctatcaccgtagctactggactcgcaggtgctggtggctctggtggaagcagtaatggtgccattggaagtagcagtatggatctcgatgacttggacacagatttcgacgacatcgataccgctgatctcctttcccctttgtga

SEQ ID NO: 20 - protein, 343 aa, Arabidopsis thaliana
MERYEIVKDIGSGNFGVAKLVRDKFSKELFAVKFIERGQKIDEHVQREIMNHRSLIHPNIIRFKEVLLTATHLALVMEYAAGGELFGRICSAGRFSEDEARFFFQQLISGVNYCHSLQICHRDLKLENTLLDGSEAPRVKICDFGYSKSGVLHSQPKTTVGTPAYIAPEVLSTKEYDGKIADVWSCGVTLYVMLVGAYPFEDPSDPKDFRKTIGRILKAQYAIPDYVRVSDECRHLLSRIFVANPEKRITIEEIKNHSWFLKNLPVEMYEGSLMMNGPSTQTVEEIVWIIEEARKPITVATGLAGAGGSGGSSNGAIGSSSMDLDDLDTDFDDIDTADLLSPL

SEQ ID NO: 21 - DNA, 1089 bp, Arabidopsis thaliana
atggatccggcgactaattcaccgattatgccgattgatttaccgattatgcacgacagtgatcgttacgacttcgttaaagatattggctctggtaatttcggcgttgctcgtctcatgaccgatagagtcaccaaggagcttgttgctgttaaatacatcgagagaggagaaaagattgatgaaaatgttcagagggagattatcaatcatagatcattgagacatcctaatattgttaggtttaaagaggtgattttgacgccttcccatttggctattgttatggaatatgctgctggtggagaactttatgagcggatttgtaatgccggacggtttagtgaagatgaggctcggttcttctttcagcagcttatatctggagttagctattgtcatgcaatgcaaatatgccatcgggatctgaagctggaaaatacattgttagatggaagtccggcacctcgtttgaaaatatgtgattttggttattccaagtcttctgttcttcattcccaaccaaagtcaactgttggtactcctgcatacattgcaccagagattcttcttcgacaggaatatgatggcaagcttgcagatgtatggtcttgcggtgtaacattatatgtaatgttggttggagcttatccattcgaggatccacaggagccacgagattatcgaaagacaatacaaagaatccttagtgtcacatactcgatcccagaggacttacacctctcaccagaatgtcgccatctaatatcgaggatcttcgtggctgatccggcaacaagaatcactattccggagatcacatccgataaatggttcttgaagaatctaccaggtgatttgatggatgagaaccgaatgggaagtcagtttcaagagcctgagcagccaatgcagagccttgacacgattatgcagataatatcggaggctacgattccgactgttcgtaatcgttgcctcgatgatttcatggcggataatcttgatctagacgatgacatggatgactttgattccgaatctgagattgatgttgacagtagtggagagatagtttatgctctctga

SEQ ID NO: 22 - protein, 362 aa, Arabidopsis thaliana
MDPATNSPIMPIDLPIMHDSDRYDFVKDIGSGNFGVARLMTDRVTKELVAVKYIERGEKIDENVQREIINHRSLRHPNIVRFKEVILTPSHLAIVMEYAAGGELYERICNAGRFSEDEARFFFQQLISGVSYCHAMQICHRDLKLENTLLDGSPAPRLKICDFGYSKSSVLHSQPKSTVGTPAYIAPEILLRQEYDGKLADVWSCGVTLYVMLVGAYPFEDPQEPRDYRKTIQRILSVTYSIPEDLHLSPECRHLISRIFVADPATRITIPEITSDKWFLKNLPGDLMDENRMGSQFQEPEQPMQSLDTIMQIISEATIPTVRNRCLDDFMADNLDLDDDMDDFDSESEIDVDSSGEIVYAL

SEQ ID NO: 23 - DNA, 1053 bp, Arabidopsis thaliana
atggagagatacgacatcttaagagatcttggttccggtaactttggagttgctaagcttgtcagagaaaaagccaacggagagttttacgccgttaaatacatcgaaagaggccttaagattgatgaacatgttcagagagagatcataaaccacagagacttgaagcatcctaatatcatcagatttaaagaggtttttgtaacaccaacacatcttgccatagtaatggagtatgcagctggtggtgaactttttgaaagaatttgcaatgccggtagattcagcgaagacgaaggaagatattatttcaaacaacttatctcgggagttagctattgtcacgctatgcaaatatgtcacagagaccttaagctcgagaatacactcttagacgggagcccgtcgtcgcatcttaaaatatgtgattttggatactccaagtcatcagttttacactctcaaccaaaatccaccgtgggaactccggcttacgttgctccggaagtcttgtcccggaaagaatataatggaaagattgcagatgtgtggtcgtgtggggtgaccttatatgtaatgttagttggtgcttatccctttgaagatcccgaagatccacggaacattagaaacaccattcagaggatattaagtgtacactacaccataccggattacgtcaggatttcctccgagtgcaagcatctcttgtctcgtatctttgtggctgaccctgataagagaataactgtaccggaaatcgaaaagcacccgtggttcttgaagggccctttggttgtgccgccggaggaagagaaatgcgataatggagttgaagaagaagaagaagaagaagagaagtgtcgacagagtgttgaagagatagtgaagataatagaggaagcaagaaagggagtaaatggtacggataataatggtggattagggttaatagatgggagcattgatcttgatgatattgatgatgctgatatttatgatgatgttgatgatgatgaggagagaaatggtgatttcgtatgtgctctatga

SEQ ID NO: 24 - protein, 350 aa, Arabidopsis thaliana
MERYDILRDLGSGNFGVAKLVREKANGEFYAVKYIERGLKIDEHVQREIINHRDLKHPNIIRFKEVFVTPTHLAIVMEYAAGGELFERICNAGRFSEDEGRYYFKQLISGVSYCHAMQICHRDLKLENTLLDGSPSSHLKICDFGYSKSSVLHSQPKSTVGTPAYVAPEVLSRKEYNGKIADVWSCGVTLYVMLVGAYPFEDPEDPRNIRNTIQRILSVHYTIPDYVRISSECKHLLSRIFVADPDKRITVPEIEKHPWFLKGPLVVPPEEEKCDNGVEEEEEEEEKCRQSVEEIVKIIEEARKGVNGTDNNGGLGLIDGSIDLDDIDDADIYDDVDDDEERNGDFVCAL

SEQ ID NO: 25 - DNA, 1029 bp, Oryza sativa
atggagcggtacgaggtgatgagggacatcgggtccgggaacttcggggtggccaagctcgtccgcgacgtcgccaccaaccacctcttcgccgtcaagttcatcgagaggggactcaagattgatgaacatgttcaaagggagattatgaaccaccgatcactgaagcatccaaacataatccggttcaaggaggtcgtgctaactcccacacatttggcaatagttatggaatatgctgctggtggtgagctatttgaaaggatttgcaacgcagggagattcagtgaggatgaggcaaggttcttcttccaacagctgatttctggagtgagctattgtcattctatgcaagtatgccatagagatttgaaactcgaaaatactctcttggatggcagtgtcacacctcggcttaagatttgtgattttggttactccaagtcttctgtcctgcactctcaaccgaaatcaactgttggcacaccggcttacattgctccagaggtcctctctagaaaggaatacgatggaaaggtagctgatgtttggtcatgtggggtaacactctatgtgatgcttgttggtgcgtatccttttgaggaccctgatgacccaaggaacttccgcaagacgatcactaggatactcagtgtacagtattcaattccagactacgttcgagtttcagcggactgcagacatctcctgtcccggattttcgttggaaatcctgagcagaggataactatcccagagatcaagaaccacccatggttcctgaagaacctgcccatcgagatgaccgacgagtaccagaggagcatgcagctggcggacatgaacacgccgtcgcagagcctggaggaggtcatggcgatcattcaggaggcccggaaaccgggcgacgccatgaagctcgccggcgccgggcaggtcgcctgcctggggagcatggatctcgacgacatcgacgatatcgacgacattgacatcgagaacagcggggacttcgtgtgcgccttgtga

SEQ ID NO: 26 - protein, 342 aa, Oryza sativa
MERYEVMRDIGSGNFGVAKLVRDVATNHLFAVKFIERGLKIDEHVQREIMNHRSLKHPNIIRFKEVVLTPTHLAIVMEYAAGGELFERICNAGRFSEDEARFFFQQLISGVSYCHSMQVCHRDLKLENTLLDGSVTPRLKICDFGYSKSSVLHSQPKSTVGTPAYIAPEVLSRKEYDGKVADVWSCGVTLYVMLVGAYPFEDPDDPRNFRKTITRILSVQYSIPDYVRVSADCRHLLSRIFVGNPEQRITIPEIKNHPWFLKNLPIEMTDEYQRSMQLADMNTPSQSLEEVMAIIQEARKPGDAMKLAGAGQVACLGSMDLDDIDDIDDIDIENSGDFVCAL

SEQ ID NO: 27 - DNA, 1020 bp, Oryza sativa
atggagaggtacgaggtgatcaaggacatagggtcggggaacttcggcgtggccaagcttgtccgggatgtgcggaccaaggagctgtttgccgtcaagttcatcgagagggggcagaagatcgacgagaatgtccaaagggagattatgaaccacaggtcactgaggcatccgaacattgttagattcaaggaggttgtgctaactcccacacatttggccatagttatggaatatgctgctggaggtgagctattcgaaaggatttgcagtgctgggaggtttagcgaggatgaggcaaggttcttcttccagcagttgatttcaggagttagctactgtcattccatgcaaatatgtcatagagatttgaaactagaaaatactctcttggatgggagcatagcacctcggctcaagatatgtgattttggttactcaaagtcctctttgttgcactctcaaccgaaatctactgtcggtactccagcttatatcgctcctgaggtccttgctagaaaagaatatgatggaaaggttgctgacgtttggtcatgtggagtaactctatatgtgatgcttgttggtgcgtacccctttgaggaccctgacgaaccaagaaacttccgcaagacaattactcggatactaagcgtacaatacatggttcctgattatgttcgagtttcgatggaatgcagacatcttctgtcccggattttcgtggcaaacccagagcaacgaattaccattcctgagatcaagaaccacccatggttcctcaagaacctgccgatcgagatgactgacgagtaccagatgagcgtccagatgaacgacatcaacaccccgtcacagggcctggaggagatcatggccatcatacaggaggcgcggaagccgggtgatggctccaaattctccgggcagatcccgggcctagggagcatggagctcgacgacgttgacaccgacgacatcgacgtcgaggacagcggcgacttcgtgtgcgcattgtga

SEQ ID NO: 28 - protein, 339 aa, Oryza sativa
MERYEVIKDIGSGNFGVAKLVRDVRTKELFAVKFIERGQKIDENVQREIMNHRSLRHPNIVRFKEVVLTPTHLAIVMEYAAGGELFERICSAGRFSEDEARFFFQQLISGVSYCHSMQICHRDLKLENTLLDGSIAPRLKICDFGYSKSSLLHSQPKSTVGTPAYIAPEVLARKEYDGKVADVWSCGVTLYVMLVGAYPFEDPDEPRNFRKTITRILSVQYMVPDYVRVSMECRHLLSRIFVANPEQRITIPEIKNHPWFLKNLPIEMTDEYQMSVQMNDINTPSQGLEEIMAIIQEARKPGDGSKFSGQIPGLGSMELDDVDTDDIDVEDSGDFVCAL

SEQ ID NO: 29 - DNA, 1005 bp, Oryza sativa
atggaggagaggtacgaggcgttgaaggagctcggggccggcaacttcggggtggccaggctggtcagggacaagaggagcaaggagctcgtcgccgtcaagtacatcgagaggggcaagaagattgatgaaaatgtgcagagggagatcatcaatcataggtcgctccggcatcccaatatcattcggtttaaggaggtttgtttgacacccacacacctagccattgtcatggagtatgctgctggtggagaactctttgaacaaatctgcaccgcagggcgattcagcgaagacgaggcaaggtacttcttccagcagctaatatcaggtgtcagctactgtcattctctggaaatttgccaccgtgatcttaaacttgagaacaccctcctggatggaagcccaacacctcgtgtgaagatttgtgactttggttactcaaagtctgctttgctgcattcgaagccgaagtctacagttggtactccagcatacatagcgccagaagttctttcaagagaagaatatgatggcaaggtagcagacgtttggtcctgtggtgtgacactgtacgtgatgcttgtcggttcatacccgtttgaagatccaggtgatccgaggaatttccgcaaaacgatcagcagaattcttggcgtgcaatactccatcccggactacgtgagggtgtcttccgactgcaggcgccttctatctcaaatatttgttgccgatccttcaaagaggatcacgatccctgagataaagaagcacacgtggtttctgaagaatctgccaaaggagatatcggagagggagaaggccgactacaaggacacggacgccgcccctccgacgcaggccgtcgaggagatcatgcggatcatccaggaggccaaggtccccggcgacatggccgccgccgacccggcgctgctcgcggagctcgccgagctgaagagcgacgacgaagaggaggccgccgatgagtatgacacctactga

SEQ ID NO: 30 - protein, 334 aa, Oryza sativa
MEERYEALKELGAGNFGVARLVRDKRSKELVAVKYIERGKKIDENVQREIINHRSLRHPNIIRFKEVCLTPTHLAIVMEYAAGGELFEQICTAGRFSEDEARYFFQQLISGVSYCHSLEICHRDLKLENTLLDGSPTPRVKICDFGYSKSALLHSKPKSTVGTPAYIAPEVLSREEYDGKVADVWSCGVTLYVMLVGSYPFEDPGDPRNFRKTISRILGVQYSIPDYVRVSSDCRRLLSQIFVADPSKRITIPEIKKHTWFLKNLPKEISEREKADYKDTDAAPPTQAVEEIMRIIQEAKVPGDMAAADPALLAELAELKSDDEEEAADEYDTY

SEQ ID NO: 31 - DNA, 1083 bp, Oryza sativa
atggagaagtacgaggcggtgagggacatcgggtcggggaacttcggggtggcgcggctgatgcgcaaccgcgagacccgcgagctcgtcgccgtcaagtgcatcgagcgcggccaccggatagatgagaatgtgtacagggagatcatcaaccaccgctcgctgcgccaccccaacatcattcgcttcaaggaggtgatactgacgccaacgcatcttatgattgtcatggagttcgcagcaggcggggagctgttcgatcgaatctgtgatcgtggacggttcagtgaggatgaggccaggtatttctttcagcagctgatctgtggagtgagctactgccatcacatgcaaatatgccatagagatttgaagttggagaatgttctcttggatggcagcccagctccacggcttaagatatgtgattttggctactccaagtcatcagtattgcattcaagacccaaatcagcagtggggacgccagcatatatcgcaccagaggtgctatcccgccgtgagtatgatggaaagcttgcagatgtatggtcctgtggtgtgactctttacgtcatgcttgtgggagcctacccatttgaagaccaggacgaccccaagaacattcgcaaaaccattcagagaataatgtcagtgcaatataagataccagattacgtccacatatctgcagaatgcaaacagcttattgcccgcatttttgtcaacaatccattgaggagaatcacgatgaaggaaataaagagccacccgtggttcttgaagaacctccccagggagctcacggagactgcgcaagccatgtactacaggagggacaactccgtgccttccttctcagaccagacctcagaagagatcatgaagattgttcaagaagcaagaaccatgccgaaatcatccaggacaggctactggagcgacgcgggttcagacgaggaggagaaggaagaggaagagaggccagaagagaacgaggaagaggaggaagatgagtacgataagagggtcaaagaggtccatgcgagcggggagctccgtatgagctcactgcgcatatga

SEQ ID NO: 32 - protein, 360 aa, Oryza sativa
MEKYEAVRDIGSGNFGVARLMRNRETRELVAVKCIERGHRIDENVYREIINHRSLRHPNIIRFKEVILTPTHLMIVMEFAAGGELFDRICDRGRFSEDEARYFFQQLICGVSYCHHMQICHRDLKLENVLLDGSPAPRLKICDFGYSKSSVLHSRPKSAVGTPAYIAPEVLSRREYDGKLADVWSCGVTLYVMLVGAYPFEDQDDPKNIRKTIQRIMSVQYKIPDYVHISAECKQLIARIFVNNPLRRITMKEIKSHPWFLKNLPRELTETAQAMYYRRDNSVPSFSDQTSEEIMKIVQEARTMPKSSRTGYWSDAGSDEEEKEEEERPEENEEEEEDEYDKRVKEVHASGELRMSSLRI

SEQ ID NO: 33 - DNA, 1113 bp, Oryza sativa
atggagaaatacgagccagttcgggagatcggggcgggcaacttcggggtagcgaagctgatgcggaacaaggagacgcgggagctggtggcgatgaagttcatcgagagagggaacaggatcgacgagaacgtgttccgggagatcgtgaatcatcgttcgctgcgtcacccgaacataataaggttcaaggaggtggtggtgacggggaggcatctggcgatcgtgatggagtacgcggcgggaggggagctgttcgagaggatatgcgaggcggggaggttccacgaggacgaggcgcgctacttcttccagcagctggtgtgcggggtgagctactgccacgccatgcagatctgccaccgcgacctcaagctggagaatacgctgctggacggcagcccggccccgcgcctcaagatctgcgacttcggctactccaagtcctccctcctccactcccgccccaaatccaccgtcggcacccccgcctacatcgcccccgaggtcctctcccgccgcgagtacgacggcaagctcgccgacgtctggtcctgcggcgtcaccctctacgtcatgctcgtcggcgcttaccctttcgaggatcccaaggaccccaagaacttcagaaagaccatctcgcgcatcatgtccgtccagtacaagatccccgagtacgtccacgtctcccagccctgccgccacctcctctcccgcatcttcgtcgccaacccctacaagcgcatcagcatgggcgagatcaagagccacccctggttcctcaagaacctgccgcgcgagctcaaggaggaggcgcaggccgtctactacaaccgccggggagccgatcacgcggcttccagcgcaagtagtgcggctgctgcagctgccttctcgccgcagagcgtggaggacatcatgaggatcgtgcaggaggcgcagaccgtccccaagcccgacaagcccgtctctggctacggctggggcaccgacgacgacgacgacgaccaacaaccagctgaggaggaggacgaagaagacgactacgacaggacggtgcgcgaggttcacgccagcgtcgacctcgacatgtcaaacctccaaatctcctga

SEQ ID NO: 34 - protein, 370 aa, Oryza sativa
MEKYEPVREIGAGNFGVAKLMRNKETRELVAMKFIERGNRIDENVFREIVNHRSLRHPNIIRFKEVVVTGRHLAIVMEYAAGGELFERICEAGRFHEDEARYFFQQLVCGVSYCHAMQICHRDLKLENTLLDGSPAPRLKICDFGYSKSSLLHSRPKSTVGTPAYIAPEVLSRREYDGKLADVWSCGVTLYVMLVGAYPFEDPKDPKNFRKTISRIMSVQYKIPEYVHVSQPCRHLLSRIFVANPYKRISMGEIKSHPWFLKNLPRELKEEAQAVYYNRRGADHAASSASSAAAAAAFSPQSVEDIMRIVQEAQTVPKPDKPVSGYGWGTDDDDDDQQPAEEEDEEDDYDRTVREVHASVDLDMSNLQIS

SEQ ID NO: 35 - DNA, 1098 bp, Oryza sativa
atggagaagtacgagctgctcaaggacatcgggtcgggcaacttcggtgtggcgcggctgatgcggaacagggagaccaaggagctcgtcgccatgaagtacataccgcgtggcctcaagattgacgagaatgtggcgagggagatcataaaccaccgctcgctgcggcacccaaacatcatccggttcaaggaggtcgtgctcacgcctacccacctcgcgatcgtcatggagtacgccgccggcggcgagctgttcgaccggatctgcagcgccgggagattcagcgaggacgagtcgaggtatttcttccagcaactaatttgcggcgtcagctactgccacttcatgcaaatttgccaccgggatttgaagctggagaacacgctgctggatggcagccctgcgccgcgcctcaagatctgcgactttggctactccaagtcatcactgctgcactcaaagccgaagtcgacggtcgggactcccgcgtacatcgctccggaggtgctctctcgccgggagtatgacggcaagatggcagatgtatggtcttgtggggtgaccctttatgtgatgctcgtcggtgcttacccttttgaggacccagatgatcccaagaatttcagaaaaacaatcgggagaatcgtatcaattcagtacaaaataccagagtacgtccatatatcccaagattgtagacaactcctctctcgaatctttgtcgcgaatcctgcaaagagaataacaataagagagatcagaaaccacccttggtttatgaagaacttgccgcgggagcttacagaagcggcgcaagcgaagtactacaagaaggacaacagtgcccgtacattctcggatcagaccgtcgacgagatcatgaagattgtacaagaggcaaagacaccacctccatcgtcgactccagtggccggtttcggttggaccgaggaagaagagcaggaggacggtaagaatcccgacgacgacgagggagacagggatgaggaggagggcgaggaaggcgatagcgaggacgagtacaccaagcaggtgaagcaagcccatgccagctgtgacttgcagaagagctga

SEQ ID NO: 36 - protein, 365 aa, Oryza sativa
MEKYELLKDIGSGNFGVARLMRNRETKELVAMKYIPRGLKIDENVAREIINHRSLRHPNIIRFKEVVLTPTHLAIVMEYAAGGELFDRICSAGRFSEDESRYFFQQLICGVSYCHFMQICHRDLKLENTLLDGSPAPRLKICDFGYSKSSLLHSKPKSTVGTPAYIAPEVLSRREYDGKMADVWSCGVTLYVMLVGAYPFEDPDDPKNFRKTIGRIVSIQYKIPEYVHISQDCRQLLSRIFVANPAKRITIREIRNHPWFMKNLPRELTEAAQAKYYKKDNSARTFSDQTVDEIMKIVQEAKTPPPSSTPVAGFGWTEEEEQEDGKNPDDDEGDRDEEEGEEGDSEDEYTKQVKQAHASCDLQKS

SEQ ID NO: 37 - DNA, 1080 bp, Oryza sativa
atggagaggtacgagctgctcaaggacatcggcgccgggaacttcggggtggcgcggctgatgcggaataaggagaccaaggagctggtcgccatgaagtacatccctcggggcctcaagattgacgagaatgtggcgagggagatcatcaaccaccggtcgctgcggcaccccaacatcatccgcttcaaggaggtggtggtcacgccgacgcacctggcgatcgtgatggagtacgctgccggcggcgagttgttcgaccggatctgcaacgccgggaggttcagcgaggacgaggccaggtatttcttccagcagctcatctgcggcgtgagctactgccacttcatgcaaatttgccaccgggatttgaagctggagaacacgctgctggacggcagcccggcgccccgcctcaagatctgcgacttcggttactccaagtcgtcgctgctgcactcgaagcccaagtcgacggtcgggacgccggcgtacatcgcgccggaggtgctatcccgccgggagtacgacggcaagacagccgatgtgtggtcttgtggagtgactctttatgtgatgcttgttggtgcttacccctttgaggaccctgatgaccccaagaatttcagaaagaccattgggagaataatgtcaattcagtacaaaatacccgagtacgtccatgtatcccaggactgcaggcaactcctttctagaatttttgttgcaaaccctgcaaagagaataacaataagggagatcaggaaccacccatggttcctgaagaacctgccaagagagctcacagaagctgcacaggcaatgtactacaagaaggataacagtgccccgacctactccgtccagtcggtcgaggagatcatgaagattgtcgaggaagcgcggacgccgcctcggtcctccacccccgtggccggctttggctggcaagaggaggatgagcaggaggacaacagcaagaagccagaggaagaacaggaggaagaggaagatgctgaggatgagtacgacaagcaggtgaaacaagtccatgccagtggtgagtttcagctcagctga

SEQ ID NO: 38 - protein, 359 aa, Oryza sativa
MERYELLKDIGAGNFGVARLMRNKETKELVAMKYIPRGLKIDENVAREIINHRSLRHPNIIRFKEVVVTPTHLAIVMEYAAGGELFDRICNAGRFSEDEARYFFQQLICGVSYCHFMQICHRDLKLENTLLDGSPAPRLKICDFGYSKSSLLHSKPKSTVGTPAYIAPEVLSRREYDGKTADVWSCGVTLYVMLVGAYPFEDPDDPKNFRKTIGRIMSIQYKIPEYVHVSQDCRQLLSRIFVANPAKRITIREIRNHPWFLKNLPRELTEAAQAMYYKKDNSAPTYSVQSVEEIMKIVEEARTPPRSSTPVAGFGWQEEDEQEDNSKKPEEEQEEEEDAEDEYDKQVKQVHASGEFQLS

SEQ ID NO: 39 - DNA, 1116 bp, Oryza sativa
atggcagcggcgggggccggggcgggggcgccggatcgggcggcgctgacggtgggcccggggatggacatgccgatcatgcacgacagcgaccggtacgagctcgtgcgcgacatcggctccggcaacttcggcgtcgcccgcctcatgcgcgaccgccgcaccatggagctcgtcgccgtcaagtacatcgagcgcggcgagaagatagatgataatgtccagcgtgagattataaatcaccgatcgttgaaacatcctaacattattaggtttaaggaggttattttaaccccaactcatcttgctattgtcatggaatatgcctctggtggtgagcttttcgagagaatttgtaagaatgtacggttcagtgaagatgaggctcgctacttcttccagcagcttatctcgggagtcagctactgccattcaatgcaagtatgccaccgtgatttgaagttggagaatacactgctggatggaagccctgctccacgcttgaaaatatgtgactttggctattctaagtcttcagttctccattcacaaccaaaatccactgtaggaacccctgcttatattgcacctgaagttctgttgaagaaagaatacgatggcaagactgctgatgtatggtcctgtggtgtgactctatatgttatggtagttggtgcatatcctttcgaggatccagaagagcctaagaacttccgtaaaacaattcagcgtatcttgaatgttcagtactcaattccagaaaacgtggacatatctccagaatgtaggcatctaatttcgaggatttttgtcggggatccgtctttgaggataacaatcccagaaatacggagccatggctggttcttgaagaaccttcctgcagatttgatggacgatgatagtatgagcagccagtatgaggaacctgatcagccaatgcaaaccatggatcagatcatgcaaattttaactgaggccaccataccacctgcttgctctcgaataaaccacatcctaactgatggactcgacctagacgatgacatggatgacctcgattccgactcagatattgatgttgatagcagcggcgagatcgtctatgcgatgtaa

SEQ ID NO: 40 - protein, 371 aa, Oryza sativa
MAAAGAGAGAPDRAALTVGPGMDMPIMHDSDRYELVRDIGSGNFGVARLMRDRRTMELVAVKYIERGEKIDDNVQREIINHRSLKHPNIIRFKEVILTPTHLAIVMEYASGGELFERICKNVRFSEDEARYFFQQLISGVSYCHSMQVCHRDLKLENTLLDGSPAPRLKICDFGYSKSSVLHSQPKSTVGTPAYIAPEVLLKKEYDGKTADVWSCGVTLYVMVVGAYPFEDPEEPKNFRKTIQRILNVQYSIPENVDISPECRHLISRIFVGDPSLRITIPEIRSHGWFLKNLPADLMDDDSMSSQYEEPDQPMQTMDQIMQILTEATIPPACSRINHILTDGLDLDDDMDDLDSDSDIDVDSSGEIVYAM

SEQ ID NO: 41 - DNA, 1086 bp, Oryza sativa
atggagagggcggcggcggggccgctggggatggagatgccgataatgcacgacggtgacaggtacgagctggtgaaggagatcgggtcggggaacttcggcgtcgcccgcctcatgcgcaaccgcgcctccggcgacctcgtcgccgtcaagtacatcgaccgcggcgagaagattgacgagaacgtgcagagggagatcatcaaccacaggtcgctgcgccaccccaacatcatccgattcaaggaggttattctgacgccgacgcatctcgcgatcgtcatggagtacgcctccggcggcgagctcttcgagcgcatctgcagcgccggccgcttcagcgaggacgaggctcgtttcttcttccagcagctgatatctggagttagctactgccattccatgcaagtatgccatcgtgacttaaagctggagaacactctgctagatggaagtactgctcctcgcttgaagatatgtgactttggttactcgaagtcatcggttcttcattcacaaccaaaatcaacagttggaactccagcttatattgctccagaagttttgctcaagaaagaatacgatggaaagattgccgatgtttggtcatgcggtgtgacgctctacgtgatgttggttggcgcataccctttcgaggatcctgaagatcccaagaacttcagaaagacaattcagaaaatattgggtgttcagtactcaattccagactatgtccacatatctccggagtgccgcgatctcattacgaggatttttgttggcaacccagctagtaggatcaccatgcctgagataaagaaccacccatggttcatgaagaacatcccggctgacctcatggatgatggcatggttagcaatcagtacgaggagcctgaccagccgatgcagaatatgaacgagatcatgcagatactggcagaagcaacaattccagcagcaggcaccagtggaatcaatcagttcttgactgacagccttgacctcgacgacgacatggaggatatggactcggaccttgaccttgacattgagagcagcggagagatcgtatatgccatgtaa

SEQ ID NO: 42 - protein, 361 aa, Oryza sativa
MERAAAGPLGMEMPIMHDGDRYELVKEIGSGNFGVARLMRNRASGDLVAVKYIDRGEKIDENVQREIINHRSLRHPNIIRFKEVILTPTHLAIVMEYASGGELFERICSAGRFSEDEARFFFQQLISGVSYCHSMQVCHRDLKLENTLLDGSTAPRLKICDFGYSKSSVLHSQPKSTVGTPAYIAPEVLLKKEYDGKIADVWSCGVTLYVMLVGAYPFEDPEDPKNFRKTIQKILGVQYSIPDYVHISPECRDLITRIFVGNPASRITMPEIKNHPWFMKNIPADLMDDGMVSNQYEEPDQPMQNMNEIMQILAEATIPAAGTSGINQFLTDSLDLDDDMEDMDSDLDLDIESSGEIVYAM

SEQ ID NO: 43 - DNA, 1089 bp, Oryza sativa
atggaccgggcggcgctgacggtggggccggggatggacatgccgataatgcacgacggcgaccggtacgagctggtgcgggacatcggctccggcaacttcggcgtcgcgcgcctcatgcgcagccgcgccgacggccagctcgtcgccgtcaagtacatcgagcgcggcgacaagatcgacgagaacgtgcagcgggagatcatcaaccaccgctcgctgcgccaccccaacatcatccgcttcaaggaggtcatcctcacccccacccacctcgccatcgtcatggagtacgcctccggcggcgagctcttcgagcgtatctgcaacgccggcaggttcagcgaggacgaggcacggttctttttccagcaactgatttcaggagtcagctattgccattccatgcaagtatgccatcgtgacctgaagctggagaacaccctgctcgacggcagcacggcgcctcgcctcaagatatgcgactttggctattcaaagtcgtctgttcttcattcgcaaccaaaatctactgttggaactccggcatacatcgctcctgaggttctgctgaagaaggaatatgatggaaagattgctgatgtgtggtcgtgtggagtaaccctctacgtaatgctggttggtgcatatccttttgaggatccagatgagcctaagaatttcaggaagacaattcagagaatattgggtgtgcagtactctattccagattatgtccacatatctccagagtgccgagatcttattgcgaggatttttgtggccaacccagccactagaatctctatccccgagatcagaaatcatccatggttcttgaagaatctcccagctgaccttatggatgatagcaagatgagcagccagtacgaggagcccgaacagccaatgcagagcatggatgagatcatgcagatactggcagaggcgaccataccagcagctgggtctggtggaatcaaccagttcttgaatgatggccttgacctcgatgatgacatggaggaccttgattcagaccccgatcttgacgtggaaagcagtggggagatagtatacgctatgtga

SEQ ID NO: 44 - protein, 362 aa, Oryza sativa
MDRAALTVGPGMDMPIMHDGDRYELVRDIGSGNFGVARLMRSRADGQLVAVKYIERGDKIDENVQREIINHRSLRHPNIIRFKEVILTPTHLAIVMEYASGGELFERICNAGRFSEDEARFFFQQLISGVSYCHSMQVCHRDLKLENTLLDGSTAPRLKICDFGYSKSSVLHSQPKSTVGTPAYIAPEVLLKKEYDGKIADVWSCGVTLYVMLVGAYPFEDPDEPKNFRKTIQRILGVQYSIPDYVHISPECRDLIARIFVANPATRISIPEIRNHPWFLKNLPADLMDDSKMSSQYEEPEQPMQSMDEIMQILAEATIPAAGSGGINQFLNDGLDLDDDMEDLDSDPDLDVESSGEIVYAM

SEQ ID NO: 45 - DNA, 1080 bp, Brassica napus
atggagaagtacgagctggtgaaagacataggagctgggaatttcggagtggcgaggctcatgaaggtcaaagactctaaggagctcgttgccatgaagtacatcgagcgtggtcccaagattgatgagaacgtggcaagagagatttataatcacagatcgcttcgccatcctaatattatccgctttaaggaggtggtgttgactccgactcatcttgctattgccatggagtatgctgctggtggtgaacttttcgagcgtatatgcggtgctggaagattcagtgaggatgaggcgagatacttcttccagcagcttatatcaggtgttagctattgccatgctatgcaaatatgccatagagatctgaagctcgagaatacactccttgatggaagtcctgctccacgtctcaaaatctgtgattttggttattccaagtcctctctactgcactcgaggcctaaatcaactgttggaactccagcatatattgcacctgaggtcctctctcggagagaatatgatggcaagatggctgatgtatggtcctgtggtgttactctttatgtcatgcttgttggagcatacccttttgaagaccaggaagaccccaaaaacttcaggaaaacaatacaaaaaatcatggctgttcagtacaagatcccggactacgtccacatctcacaagattgcaaacatctcctttcccgtatatttgtggccaactcactcaagaggataaccattgcggaaatcaagaaacacccatggttcacgaagaacttgccaagggagctcacagagacagctcaagctgcatatttcaagaaagagaatccaaccttctccgcccagaccgctgaagagatcatgaagatagtggatgacgccaaaacgcctccgcctgtttcccgttccattggaggttttggctggggaggagagggagatttagaggggaaagaggaagaggaggtggatgaagaggaggttgaggaagaggaagacgaagaagatgaatatgataagactgtaaaggaagtacacgcaagcggagaagtgagaatcagttga

SEQ ID NO: 46 - protein, 359 aa, Brassica napus
MEKYELVKDIGAGNFGVARLMKVKDSKELVAMKYIERGPKIDENVAREIYNHRSLRHPNIIRFKEVVLTPTHLAIAMEYAAGGELFERICGAGRFSEDEARYFFQQLISGVSYCHAMQICHRDLKLENTLLDGSPAPRLKICDFGYSKSSLLHSRPKSTVGTPAYIAPEVLSRREYDGKMADVWSCGVTLYVMLVGAYPFEDQEDPKNFRKTIQKIMAVQYKIPDYVHISQDCKHLLSRIFVANSLKRITIAEIKKHPWFTKNLPRELTETAQAAYFKKENPTFSAQTAEEIMKIVDDAKTPPPVSRSIGGFGWGGEGDLEGKEEEEVDEEEVEEEEDEEDEYDKTVKEVHASGEVRIS

SEQ ID NO: 47 - DNA, 1065 bp, Brassica napus
atggagaagtacgagctggtgaaagacataggcgctgggaatttcggagtggcgaggctcatgaaggtcaaaaactctaaagagcttgttgccatgaagtacatcgagcgtggtcccaagattgatgagaatgtggcaagagagatcattaatcacagatcgcttcgtcatcctaatattatccgttttaaggaggttgtgttgactccaactcatcttgctattgccatggaatatgctgctggtggtgaattattcgagcgtatatgcagtgctggaagattcagtgaggatgaggcgagatacttcttccagcagcttatatcaggtgttagctattgccatgctatgcaaatatgccatagagatctgaagctcgagaacacactcctggatggaagtcctgctccacgtctcaaaatctgtgattttggttattccaagtcctctctactgcactcgaggcccaaatccacagttggaactccagcatatattgcacctgaggtcctttctcggagagagtatgatggcaagatggctgatgtatggtcttgtggtgtaactctttatgtcatgcttgttggagcctacccattcgaagaccaggaagacccaaagaacttcaggaaaacaatacaaaaaatcatggctgttcagtacaagatcccggactacgtccacatctcacaggattgcaaacacctcctttcccgtatatttgttgccaattcactcaagaggataaccattgcagaaatcaagaaacacccatggttcctgaagaacctgccaagggagctcacagagacagctcaagctgcatatttcaagaaagagaatccaaccttctccccgcagaccgctgaagagatcatgaagatagtggatgacgccaaaacgcctccgcctgtttccagatccattggagggtttggctggggaggaaagggagacgaagaggaagaagaagtggatgaagaggaggtggtggaggaagaggaagacgaagaagatgaatatgataagactgtaaaggaagcacacgcaagtggagaagtgtga

SEQ ID NO: 48 - protein, 354 aa, Brassica napus
MEKYELVKDIGAGNFGVARLMKVKNSKELVAMKYIERGPKIDENVAREIINHRSLRHPNIIRFKEVVLTPTHLAIAMEYAAGGELFERICSAGRFSEDEARYFFQQLISGVSYCHAMQICHRDLKLENTLLDGSPAPRLKICDFGYSKSSLLHSRPKSTVGTPAYIAPEVLSRREYDGKMADVWSCGVTLYVMLVGAYPFEDQEDPKNFRKTIQKIMAVQYKIPDYVHISQDCKHLLSRIFVANSLKRITIAEIKKHPWFLKNLPRELTETAQAAYFKKENPTFSPQTAEEIMKIVDDAKTPPPVSRSIGGFGWGGKGDEEEEEVDEEEVVEEEEDEEDEYDKTVKEAHASGEV

SEQ ID NO: 49 - DNA, 1056 bp, Glycine max
atggataagtatgaggctgtcaaggatttgggagctgggaattttggggtggctaggctcatgaggaacaaggagaccaaggagcttgttgccatgaaatacatcgagcgtggccaaaagattgatgagaatgtggcaagagagattatcaaccacagatcccttcggcaccccaatataattcgcttcaaggaggtggttttgacccccacccatttggccatagtgatggagtatgcggctggaggagagctctttgagaggatatgcaatgctggcaggttcagtgaagatgaggctagatatttctttcagcagctgatttctggtgtacattactgtcatgccatgcaaatatgtcacagagatttgaagctagaaaatacccttttagatggaagccctgcaccccgcctgaaaatttgtgactttggttattccaagtcatcattacttcattcacggccaaaatcaactgttggaactccagcttatatagcaccagaggttctttccaggagggagtatgatggcaagttggctgatgtatggtcatgtggagtgactctttatgtcatgctggttggagcttatccctttgaggatcaggatgaccctaggaattttaggaaaacaattcagcgtataatggctgttcaatacaaaatccctgattatgttcacatatctcaagactgcagacacctcctttctcgtatatttgtagcaaatccattaaggaggatctctcttaaggaaattaagagccacccatggtttttaaagaatcttccaagagagctgactgaatcagctcaagctgtctattaccagagaggcaatccaagcttttcaattcaaagtgtggaggagatcatgaagattgtgggagaggcaagggaccctcctccagtatctagacctgtcaaaggttttggctgggatggcgaagaagatgaaggggaagaagacgtggaggaagaggaggacgaagaagacgagtatgacaagagggtcaaagaggttcatgcaagtggagaatttcaaatcagttaa

SEQ ID NO: 50 - protein, 351 aa, Glycine max
MDKYEAVKDLGAGNFGVARLMRNKETKELVAMKYIERGQKIDENVAREIINHRSLRHPNIIRFKEVVLTPTHLAIVMEYAAGGELFERICNAGRFSEDEARYFFQQLISGVHYCHAMQICHRDLKLENTLLDGSPAPRLKICDFGYSKSSLLHSRPKSTVGTPAYIAPEVLSRREYDGKLADVWSCGVTLYVMLVGAYPFEDQDDPRNFRKTIQRIMAVQYKIPDYVHISQDCRHLLSRIFVANPLRRISLKEIKSHPWFLKNLPRELTESAQAVYYQRGNPSFSIQSVEEIMKIVGEARDPPPVSRPVKGFGWDGEEDEGEEDVEEEEDEEDEYDKRVKEVHASGEFQIS

SEQ ID NO: 51 - DNA, 1050 bp, Glycine max
atggataagtacgaggctgttaaggatttgggagctggcaattttggggtggctaggctcatgaggaacaaggtcaccaaggagcttgtagccatgaaatacatcgagcgtggccccaagattgatgagaacgtggcaagggagattatgaaccacaggtcccttcggcatcccaatataattcgttacaaggaggtggttttgactcccacacatttagcaatagtgatggagtatgcagcaggaggagagctctttgagaggatatgcagtgctggcaggttcagtgaagatgaggctagatatttctttcagcagctgatttccggtgttcatttctgtcataccatgcaaatatgccacagagatttgaagctagaaaatacccttctagatggaagtcctgcacctcggttgaaaatttgtgacttcggttattccaagtcatctttgctgcactcacgacccaaatcaacagttggaacaccagcttacatagcaccggaagttctttctaggcgagagtatgacggaaagttggctgatgtatggtcatgtgcggtgactctttatgtcatgctggttggagcatatccctttgaggaccaggatgaccctaggaattttaggaaaacaattcagcgtataatggctgttcaatacaaaatccctgattatgttcacatatctcaagattgtaggcacctcctctctcgtatatttgttgcaaatccattgaggagaattactattaaggaaattaagaatcacccatggtttttgaggaatcttccaagggagctaactgaatctgctcaagctatctattaccagagagacagcccaaactttcaccttcaaagtgtggatgagataatgaaaattgtaggagaggcaagaaatccacctccagtatctagggctctcaaaggttttggctgggaaggtgaagaagatttggatgaagaagtggaggaagaagaggatgaagatgagtatgataagagggtcaaagaggttcatgcaagtggcgaatttcaaattagttaa

SEQ ID NO: 52 - protein, 349 aa, Glycine max
MDKYEAVKDLGAGNFGVARLMRNKVTKELVAMKYIERGPKIDENVAREIMNHRSLRHPNIIRYKEVVLTPTHLAIVMEYAAGGELFERICSAGRFSEDEARYFFQQLISGVHFCHTMQICHRDLKLENTLLDGSPAPRLKICDFGYSKSSLLHSRPKSTVGTPAYIAPEVLSRREYDGKLADVWSCAVTLYVMLVGAYPFEDQDDPRNFRKTIQRIMAVQYKIPDYVHISQDCRHLLSRIFVANPLRRITIKEIKNHPWFLRNLPRELTESAQAIYYQRDSPNFHLQSVDEIMKIVGEARNPPPVSRALKGFGWEGEEDLDEEVEEEEDEDEYDKRVKEVHASGEFQIS

SEQ ID NO: 53 - DNA, 1071 bp, Nicotiana tabacum
atggataaatacgagcttgtgaaagatatagggtcagggaattttggtgtggcaaggctgatgaggcacaaggaaaccaaagaacttgtggcaatgaaatacattgagagaggacataagattgatgagaatgtagcaagggagatcattaatcatagatcgcttcggcatccaaacataattcgattcaaggaggtgttagtgactcccactcatcttgccattgttatggaatatgcagctggtggagaactgtttgagcgcatttgcaatgcaggaaggtttagtgaagatgaggctaggtactttttccagcagcttatttcaggagttcactactgtcacaacatgcaaatatgccatagagatttgaagctggagaatacgcttcttgatggaagtccagctccacgcttgaagatatgtgattttggatactcaaagtcgtccctgttgcattcgaggccaaaatcaactgttgggactccagcttatattgctcctgaggtcctatcaagaagagaatatgatggcaagctggctgatgtttggtcatgcggggtgacactttatgtgatgctggttggggcatatccttttgaagatcaggaggatccgaagaattttaggaaaactattcaacgaataatggcggtacagtacaagattcccgactatgttcacatatcacaagattgtaggcaccttctctctcggatatttgttgctaatccagcaaggaggatcacaatcaaagaaatcaagtctcacccatggtttttgaagaatttgccgagggaattaacagaagcagcacaggcagcttattacagaagagaaaacccaacattttcacttcagagtgttgaggagatcatgaaaattgtggaagaggcaaagactcccgctccagcttcccgttcagtctcaggctttggctggggaggagaagaagaagaagaggagaaggaaggagatgtagaagaagaggaagaggatgaagaagaagaagacgaatatgaaaagcaagtgaagcaggcacatgaaagcggagaagttcgtctcacctaa

SEQ ID NO: 54 - protein, 356 aa, Nicotiana tabacum
MDKYELVKDIGSGNFGVARLMRHKETKELVAMKYIERGHKIDENVAREIINHRSLRHPNIIRFKEVLVTPTHLAIVMEYAAGGELFERICNAGRFSEDEARYFFQQLISGVHYCHNMQICHRDLKLENTLLDGSPAPRLKICDFGYSKSSLLHSRPKSTVGTPAYIAPEVLSRREYDGKLADVWSCGVTLYVMLVGAYPFEDQEDPKNFRKTIQRIMAVQYKIPDYVHISQDCRHLLSRIFVANPARRITIKEIKSHPWFLKNLPRELTEAAQAAYYRRENPTFSLQSVEEIMKIVEEAKTPAPASRSVSGFGWGGEEEEEEKEGDVEEEEEDEEEEDEYEKQVKQAHESGEVRLT

SEQ ID NO: 55 - DNA, 2193 bp, Oryza sativa
aatccgaaaagtttctgcaccgttttcaccccctaactaacaatatagggaacgtgtgctaaatataaaatgagaccttatatatgtagcgctgataactagaactatgcaagaaaaactcatccacctactttagtggcaatcgggctaaataaaaaagagtcgctacactagtttcgttttccttagtaattaagtgggaaaatgaaatcattattgcttagaatatacgttcacatctctgtcatgaagttaaattattcgaggtagccataattgtcatcaaactcttcttgaataaaaaaatctttctagctgaactcaatgggtaaagagagagattttttttaaaaaaatagaatgaagatattctgaacgtattggcaaagatttaaacatataattatataattttatagtttgtgcattcgtcatatcgcacatcattaaggacatgtcttactccatcccaatttttatttagtaattaaagacaattgacttatttttattatttatcttttttcgattagatgcaaggtacttacgcacacactttgtgctcatgtgcatgtgtgagtgcacctcctcaatacacgttcaactagcaacacatctctaatatcactcgcctatttaatacatttaggtagcaatatctgaattcaagcactccaccatcaccagaccacttttaataatatctaaaatacaaaaaataattttacagaatagcatgaaaagtatgaaacgaactatttaggtttttcacatacaaaaaaaaaaagaattttgctcgtgcgcgagcgccaatctcccatattgggcacacaggcaacaacagagtggctgcccacagaacaacccacaaaaaacgatgatctaacggaggacagcaagtccgcaacaaccttttaacagcaggctttgcggccaggagagaggaggagaggcaaagaaaaccaagcatcctcctcctcccatctataaattcctccccccttttcccctctctatataggaggcatccaagccaagaagagggagagcaccaaggacacgcgactagcagaagccgagcgaccgccttcttcgatccatatcttccggtcgagttcttggtcgatctcttccctcctccacctcctcctcacagggtatgtgcccttcggttgttcttggatttattgttctaggttgtgtagtacgggcgttgatgttaggaaaggggatctgtatctgtgatgattcctgttcttggatttgggatagaggggttcttgatgttgcatgttatcggttcggtttgattagtagtatggttttcaatcgtctggagagctctatggaaatgaaatggtttagggtacggaatcttgcgattttgtgagtaccttttgtttgaggtaaaatcagagcaccggtgattttgcttggtgtaataaaagtacggttgtttggtcctcgattctggtagtgatgcttctcgatttgacgaagctatcctttgtttattccctattgaacaaaaataatccaactttgaagacggtcccgttgatgagattgaatgattgattcttaagcctgtccaaaatttcgcagctggcttgtttagatacagtagtccccatcacgaaattcatggaaacagttataatcctcaggaacaggggattccctgttcttccgatttgctttagtcccagaattttttttcccaaatatcttaaaaagtcactttctggttcagttcaatgaattgattgctacaaataatgcttttatagcgttatcctagctgtagttcagttaataggtaatacccctatagtttagtcaggagaagaacttatccgatttctgatctccatttttaattatatgaaatgaactgtagcataagcagtattcatttggattattttttttattagctctcaccccttcattattctgagctgaaagtctggcatgaactgtcctcaattttgttttcaaattcacatcgattatctatgcattatcctcttgtatctacctgtagaagtttctttttggttattccttgactgcttgattacagaaagaaatttatgaagctgtaatcgggatagttatactgcttgttcttatgattcatttcctttgtgcagttcttggtgtagcttgccactttcaccagcaaagttc